(19) United States
(12) Patent Application Publication
(10) Pub. No.: US 2026/0207630 A1
(43) Pub. Date: Jul. 23, 2026

(54) FUROSEMIDE FORMULATIONS

(71) Applicant: scPharmaceuticals Inc., Burlington, MA (US)

(72) Inventor: Jennifer Vandiver, Burlington, MA (US)

(21) Appl. No.: 19/447,067

(22) Filed: Jan. 13, 2026

Related U.S. Application Data
(60) Provisional application No. 63/744,565, filed on Jan. 13, 2025.

(51) Int. Cl. A61K 31/635; A61K 9/00; A61K 9/08; A61K 47/02; A61K 47/10; A61K 47/12; A61K 47/18; A61K 47/22; A61K 47/26; A61K 47/32; A61K 47/40

(52) U.S. Cl. CPC A61K 31/635; A61K 9/0019; A61K 9/08; A61K 47/02; A61K 47/10; A61K 47/12; A61K 47/18; A61K 47/183; A61K 47/22; A61K 47/26; A61K 47/32; A61K 47/40

(57) ABSTRACT

Disclosed herein, in part, are liquid pharmaceutical formulations containing furosemide for delivering to a patient via subcutaneous injection. Methods of treating conditions such as congestion due to fluid overload, edema, and hypertension in a patient in need thereof are also provided.

### RELATED APPLICATIONS

This application claims priority to U.S. Provisional Application No. 63/744,565, filed Jan. 13, 2025, the content of which is incorporated in its entirety.

### BACKGROUND

Furosemide forms the cornerstone of parenteral diuretic therapy and has been in clinical use for nearly 50 years. It is the most widely used oral and parenteral diuretic used in heart failure. Its clinical safety has been established in numerous clinical studies and market experience. A formulation at physiological pH will potentially minimize the risk of necrosis, pain, or discomfort upon subcutaneous delivery. However, furosemide is less soluble with reduced stability in more acidic solutions. Other factors that can contribute to pain include solution tonicity, injection volume, and injection speed.

In order to enable subcutaneous delivery via an autoinjector, the delivered volume of the furosemide solution should be less than or equal to 1 mL. A low volume, high concentration solution of at least 40 mg/mL furosemide formulation would allow for subcutaneous delivery of therapeutic amounts of furosemide in a 1 mL injection.

To achieve physiological pH, a buffer must be used in the formulation. A solubilizer agent, such as surfactants, may be used to achieve the required solubility. The buffer used can impact the solubility of the drug since buffers can introduce counter-ions with which the drug might precipitate due to lower solubility of the complex. There can also be unexpected synergies between certain buffer and surfactant pairings. The challenge for finding an appropriate subcutaneous formulation of furosemide is identifying the combination of excipients that meet the interdependent requirements of pH, tonicity, and solubility that allow for the safe subcutaneous injection of the active ingredient. As such, there is a need for stable, concentrated formulations of furosemide to for subcutaneous delivery.

### SUMMARY

Provided herein are pharmaceutical formulations containing furosemide that are chemically and physically stable over their shelf-life, contain a sufficient concentration of furosemide (e.g., an effective amount in a suitable volume), and are at an appropriate pH and osmolality, for example, to permit subcutaneous administration (e.g., subcutaneous injection) of furosemide.

In an aspect, provided herein is a liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, and glycine.

In another aspect, provided herein is a liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, sodium tetraborate, potassium tetraborate, and glycine.

In an embodiment, the liquid pharmaceutical formulation optionally further comprises:
(iv) a pH adjuster.

In another aspect, provided herein is a unit liquid pharmaceutical formulation comprising:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, and glycine; and optionally
(iv) a pH adjuster that is potassium hydroxide or sodium hydroxide.

In another aspect, provided herein is a unit liquid pharmaceutical formulation comprising:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, sodium tetraborate, potassium tetraborate, and glycine; and optionally
(iv) a pH adjuster that is potassium hydroxide or sodium hydroxide.

In another aspect, provided herein is a method of treating congestion, edema, fluid overload, or hypertension in a subject in need thereof comprising administering to the subject the liquid pharmaceutical formulations of the present disclosure.

Also provided herein are kits for the treatment of congestion, edema, fluid overload, or hypertension comprising a liquid pharmaceutical formulation of the present disclosure.

### BRIEF DESCRIPTION OF THE DRAWINGS

FIG. 1 shows the concentration of furosemide in formulations with varying buffers with or without benzyl alcohol (BA).

FIG. 2 shows least squares means for formulations with varying buffers and solubilizers.

FIG. 3 shows the concentration of furosemide in formulations with two different pH adjusters (NaOH or KOH) with or without benzyl alcohol (BA).

FIG. 4 shows the effect of secondary solubilizers and two different pH adjusters (NaOH or KOH) on the solubility of furosemide in formulations using potassium phosphate buffer with or without benzyl alcohol (BA).

FIG. 5 shows the stability of the formulations described in FIG. 4.

### DETAILED DESCRIPTION

As generally described herein, the present disclosure provides liquid pharmaceutical formulations that have increased solubilization of furosemide, which allows for administration of furosemide at low volumes. Therefore, also provided herein are methods of treating various conditions, diseases, and disorders (e.g., congestion due to fluid overload, edema, and hypertension) in a subject in need thereof comprising administering the liquid pharmaceutical formulations of the present disclosure.

### Definitions

Listed below are definitions of various terms used to describe the liquid pharmaceutical formulations provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the liquid pharmaceutical formulations belong. Generally, the nomenclature used herein and the laboratory procedures are those well-known and commonly employed in the art.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. Stated alternatively, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

Where the use of the term “about” is before a quantitative value, the present invention also includes the specific quantitative value itself, unless specifically stated otherwise. As used herein, the term “about” refers to a ±10%, ±5%, ±3%, ±2%, or ±1% variation from the nominal value unless otherwise indicated or inferred from the context.

At various places in the present specification, values are disclosed in groups or in ranges. It is specifically intended that the description include each and every individual subcombination of the members of such groups and ranges. For example, a number in the range of 1 to 20 is specifically intended to individually disclose the integers 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, a “compound” (including a specifically named compound, e.g., furosemide) refers to the compound itself and its pharmaceutically acceptable salts unless otherwise understood from the context of the description or expressly limited to one particular form of the compound, e.g., the compound itself, or a pharmaceutically acceptable salt thereof.

As used herein, “furosemide” refers to a compound having the formula:

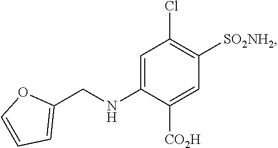 [SMILES: NS(=O)(=O)c1cc(C(=O)O)c(NCc2ccco2)cc1Cl]

or a pharmaceutically acceptable salt thereof. Such salts may include, but are not limited to, furosemide sodium salt and furosemide quaternary ammonium salt. Furosemide may be referred to by other names, for example, frusemide, 5-(aminosulphonyl)-4-chloro-2-[(2-furanyl-methyl)amino]benzoic acid, or its IUPAC name, 4-chloro-2-(furan-2-ylmethylamino)-5-sulfamoyl-benzoic acid, or its common trade name, Lasix®.

As used herein, the term “patient,” “individual” or “subject” refers to a human or a non-human mammal. Non-human mammals include, for example, livestock and pets, such as ovine, bovine, porcine, canine, feline and murine mammals. In an embodiment, the patient, subject, or individual is human.

As used herein, a “buffer” refers to an aqueous solution that is resistant to changes in pH. A buffer may include a “buffering agent” such as a weak acid and its salt, or a weak base and its salt, which assist in maintaining the stability of the pH. Examples of buffers used in pharmaceutical formulations include acetate buffers, amino acid buffers, bicarbonate buffers, carbonate buffers, citrate buffers, histidine buffers, phosphate buffers, tartrate buffers, tris(hydroxymethyl)aminomethane (or 2-amino-2-hydroxymethyl-propane-1,3-diol [(HOCH₂)₃CNH₂]) buffers, and combinations thereof. Certain of these buffers are suitable for pharmaceutical formulations administered subcutaneously.

Tris(hydroxymethyl)aminomethane or a tris(hydroxymethyl)aminomethane buffer can be referred to as “TRIS,” “Tris,” “Tris buffer,” “Trisamine,” “THAM,” “tromethamine,” and other names. In addition, many buffers and/or buffer systems can include Tris, or a pharmaceutically acceptable salt thereof, and can be used in the present teachings. For example, Tris-buffered saline (“TBS”), Tris-hydrochloride buffer (“Tris-HCl”), Tris base (pH 10.6), Tris/borate/ethylene diamine tetra-acetate (“EDTA”) buffer (“TBE”), and Tris/acetate/EDTA buffer (“TAE”). Tris base often is used with Tris-HCl to prepare Tris buffers at a desired pH. In addition, the present teachings can include Tris-related compounds, for example, compounds derived from Tris or structurally-related to Tris, that can act as a buffer.

As used herein, “tonicity” refers to the ionic strength or concentration of ions in a solution such as a pharmaceutical formulation. Tonicity often is measured in molarity (“M”). As used herein, an “isotonic solution,” an “isotonic formulation,” an “isotonic pharmaceutical formulation,” and a pharmaceutical formulation that is “isotonic” refers to a solution or formulation that has the same or similar concentration of ions as found in bodily fluids.

As used herein, “physiological pH” refers to a pH of about 7.4.

As used herein, “osmoticity,” “osmolarity,” and “osmolality” refer to the osmotic pressure of a solution such as a pharmaceutical formulation. Osmoticity often is measured in osmolarity (“Osm/L” or “OsM”) or osmolality (“Osm/kg”). When measuring freezing point depression, the observed value is the osmolality of the solution. In contrast to tonicity, osmoticity accounts for un-ionized solutes in a solution such that when present, the osmolarity or osmolality of the solution will be higher than its tonicity. The osmolarity of a liquid pharmaceutical formulation described herein can be measured, for example, using a vapor pressure method.

As used herein, “osmolarity adjustor,” “osmolarity adjustor,” and “osmotic agent” refer to a pharmaceutically acceptable compound that may be added to a liquid pharmaceutical formulation described herein in order to modulate the osmolarity of the liquid pharmaceutical formulation.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. In certain embodiments, supplementary active ingredients can also be incorporated into the pharmaceutical compositions.

As used herein, “pharmaceutically acceptable excipient” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, surfactants (e.g., polysorbate 20 or polysorbate 80), wetting agents, emulsifiers, salts for influencing osmotic pressure, buffers, coloring, and/or aromatic substances and the like that do not deleteriously react with the compounds of the invention. For examples of excipients and carriers, see Martin, Remington's Pharmaceutical Sciences, 15th Ed., Mack Publ. Co., Easton, PA (1975).

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, PA (1975).

The phrase “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66 (1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66 (1), 1-19, T. S. Wiedmann, et al., Asian J. Pharm. Sci., 2016; 11, 722-34. D. Gupta et al., Molecules, 2018, 23 (7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄⁺ (wherein W is a C₁₋₄ alkyl group), and the like.

As used herein, the term “effective amount” refers to the amount of a composition (e.g., a liquid pharmaceutical formulation of the present invention) sufficient to effect beneficial or desired results, including a desired therapeutic effect. An effective amount can be administered in one or more administrations, applications or dosages and is not intended to be limited to a particular formulation or administration route.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “congestion” (in heart failure) is the presence of signs and symptoms of extracellular fluid accumulation that results in increased cardiac filling pressures leading to reduced cardiac output. This reduced cardiac output is further exacerbated by neurohormonal activation leading to increased renal sodium and water avidity resulting in an increased plasma volume.

As used herein, “fluid overload,” “volume overload,” and “hypervolemia” may describe a medical condition where there is too much fluid in the blood. Excess fluid, primarily salt and water, may build up throughout the body resulting in weight gain.

As used herein, the term “solubilizer” refers to a substance that helps to dissolve poorly soluble substances in a solvent, such as oil-soluble or ambiphilic molecules in aqueous formulations.

As used herein, “unit liquid pharmaceutical formulation” refers to physically discrete units suited as unitary dosages for the subjects to be treated; each unit contains a predetermined quantity of active compound calculated to produce the desired therapeutic effect in association with the required pharmaceutical carrier.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### Liquid Pharmaceutical Formulations of Furosemide

As described herein, the present disclosure provides liquid pharmaceutical formulations that have increased solubilization of furosemide. Thus, in one aspect, provided herein are liquid pharmaceutical formulations comprising an effective amount of furosemide, or a pharmaceutically acceptable salt thereof, for the treatment of a condition described herein. In certain embodiments, the condition is selected from the group consisting of congestion due to fluid overload, edema, and hypertension, and combinations thereof. In certain embodiments, the condition is congestion due to fluid overload. In certain embodiments, the condition is edema. In certain embodiments, the condition is hypertension. In certain embodiments, the subject to be treated has chronic heart failure. In certain embodiments, the subject to be treated has chronic kidney disease. In certain embodiments, the condition is edema in adult patients with chronic heart failure or chronic kidney disease, including the nephrotic syndrome.

In a particular aspect, provided herein is a liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, sodium tetraborate, potassium tetraborate, and glycine.

In another aspect, provided herein is a unit liquid pharmaceutical formulation comprises:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, a cyclodextrin, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, sodium tetraborate, potassium tetraborate, and glycine; and optionally
(iv) a pH adjuster that is potassium hydroxide or sodium hydroxide.

### Furosemide

In various embodiments, provided herein are liquid pharmaceutical formulations comprising an effective amount of furosemide, or a pharmaceutically acceptable salt thereof, for the treatment of a condition described herein.

In certain embodiments, the effective amount of furosemide in the liquid pharmaceutical formulation described herein is about 40 mg to about 250 mg, about 40 mg to about 200 mg, about 80 mg to about 250 mg, about 80 mg to about 200 mg, about 40 mg to about 150 mg, about 80 mg to about 150 mg, about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, about 95 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 95 mg, about 70 mg to about 90 mg, about 70 mg to about 85 mg, about 70 mg to about 80 mg, about 70 mg to about 75 mg, about 75 mg to about 95 mg, about 75 mg to about 90 mg, about 75 mg to about 85 mg, about 75 mg to about 80 mg, about 80 mg to about 95 mg, about 80 mg to about 90 mg, about 80 mg to about 85 mg, about 85 mg to about 95 mg, about 85 mg to about 90 mg, or about 90 mg to about 95 mg.

In various embodiments, the effective amount of furosemide in a liquid pharmaceutical formulation described herein is about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, or about 150 mg.

In certain embodiments, the effective amount of furosemide in a liquid pharmaceutical formulation described herein is about 40 mg. In certain embodiments, the effective amount of furosemide in a liquid pharmaceutical formulation described herein is about 80 mg.

In various embodiments, a liquid pharmaceutical formulation described herein comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) furosemide.

In certain embodiments, a liquid pharmaceutical formulation described herein comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) furosemide.

In certain embodiments, a liquid pharmaceutical formulation described herein comprises about 5% (w/w) furosemide. In certain embodiments, a liquid pharmaceutical formulation described herein comprises about 6% (w/w) furosemide. In certain embodiments, a liquid pharmaceutical formulation described herein comprises about 7% (w/w) furosemide. In certain embodiments, a liquid pharmaceutical formulation described herein comprises about 8% (w/w) furosemide. In certain embodiments, a liquid pharmaceutical formulation described herein comprises about 9% (w/w) furosemide. In certain embodiments, a liquid pharmaceutical formulation described herein comprises about 10% (w/w) furosemide.

In certain embodiments, the concentration of furosemide in a liquid pharmaceutical formulation described herein can be 1 mg/mL to about 250 mg/mL, about 1 mg/mL to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL, about 1 mg/mL to about mg/mL mg, about 40 mg/mL to about 150 mg/mL, about 80 mg/mL to about 150 mg/mL, 1 mg/mL to about 250 mg/mL, about 1 mg/mL to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL, about 50 mg/mL to about 250 mg/mL, about 60 mg/mL to about 250 mg/mL, about 70 mg/mL to about 250 mg/mL, about 80 mg/mL to about 250 mg/mL, about 90 mg/mL to about 250 mg/mL, about 100 mg/mL to about 250 mg/mL, about 120 mg/mL to about 250 mg/mL, about 140 mg/mL to about 250 mg/mL, about 160 mg/mL to about 250 mg/mL, about 180 mg/mL to about 250 mg/mL, about 200 mg/mL to about 250 mg/mL, about 50 mg/mL to about 200 mg/mL, about 50 mg/mL to about 180 mg/mL, about 50 mg/mL to about 160 mg/mL, about 50 mg/mL to about 140 mg/mL, about 50 mg/mL to about 120 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 200 mg/mL, about 60 mg/mL to about 180 mg/mL, about 60 mg/mL to about 160 mg/mL, about 60 mg/mL to about 140 mg/mL, about 60 mg/mL to about 120 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 200 mg/mL, about 70 mg/mL to about 180 mg/mL, about 70 mg/mL to about 160 mg/mL, about 70 mg/mL to about 140 mg/mL, about 70 mg/mL to about 120 mg/mL, about 70 mg/mL to about 100 mg/mL, about 70 mg/mL to about 90 mg/mL, about 70 mg/mL to about 80 mg/mL, about 80 mg/mL to about 200 mg/mL, about 80 mg/mL to about 180 mg/mL, about 80 mg/mL to about 160 mg/mL, about 80 mg/mL to about 140 mg/mL, about 80 mg/mL to about 120 mg/mL, about 80 mg/mL to about 100 mg/mL, about 80 mg/mL to about 90 mg/mL, about 90 mg/mL to about 200 mg/mL, about 90 mg/mL to about 180 mg/mL, about 90 mg/mL to about 160 mg/mL, about 90 mg/mL to about 140 mg/mL, about 90 mg/mL to about 120 mg/mL, about 90 mg/mL to about 100 mg/mL, about 100 mg/mL to about 200 mg/mL, about 100 mg/mL to about 180 mg/mL, about 100 mg/mL to about 160 mg/mL, about 100 mg/mL to about 140 mg/mL, about 100 mg/mL to about 120 mg/mL, about 120 mg/mL to about 200 mg/mL, about 120 mg/mL to about 180 mg/mL, about 120 mg/mL to about 160 mg/mL, about 120 mg/mL to about 140 mg/mL, about 140 mg/mL to about 200 mg/mL, about 140 mg/mL to about 180 mg/mL, about 140 mg/mL to about 160 mg/mL, about 160 mg/mL to about 200 mg/mL, about 160 mg/mL to about 180 mg/mL, or about 180 mg/mL to about 200 mg/mL.

In certain embodiments, the concentration of furosemide in a liquid pharmaceutical formulation described herein can be 1 mg/mL to about 250 mg/mL, about 1 mg/ml to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL, about 1 mg/mL to about mg/mL mg, about 40 mg/mL to about 150 mg/mL, about 80 mg/mL to about 150 mg/mL, 1 mg/mL to about 250 mg/mL, about 1 mg/mL to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL.

In certain embodiments, the concentration of furosemide in a liquid pharmaceutical formulation described herein can be about 1 mg/mL to about 250 mg/mL. In certain embodiments, the concentration of furosemide in a liquid pharmaceutical formulation described herein can be about 40 mg/mL to about 200 mg/mL. In certain embodiments, the concentration of furosemide in a liquid pharmaceutical formulation described herein can be about 80 mg/mL to about 150 mg/mL.

In certain embodiments, the concentration of furosemide in a liquid pharmaceutical formulation described herein can be about 60 mg/mL to about 140 mg/mL. In certain embodiments, the concentration of furosemide in a liquid pharmaceutical formulation described herein can be about 80 mg/mL to about 120 mg/mL.

In certain embodiments, the concentration of furosemide in a liquid pharmaceutical formulation described herein can be about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, or about 250 mg/mL.

In certain embodiments, the concentration of furosemide in a liquid pharmaceutical formulation described herein is about 80 mg/mL. In certain embodiments, the concentration of furosemide in a liquid pharmaceutical formulation described herein is about 100 mg/mL. In certain embodiments, the concentration of furosemide in a liquid pharmaceutical formulation described herein is about 120 mg/mL.

In various embodiments, a liquid pharmaceutical formulation described herein may comprise a pharmaceutically acceptable salt of furosemide, e.g., furosemide sodium.

In various embodiments, a liquid pharmaceutical formulation described herein comprises a pharmaceutically acceptable salt of furosemide in an amount that provides about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, about 95 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 95 mg, about 70 mg to about 90 mg, about 70 mg to about 85 mg, about 70 mg to about 80 mg, about 70 mg to about 75 mg, about 75 mg to about 95 mg, about 75 mg to about 90 mg, about 75 mg to about 85 mg, about 75 mg to about 80 mg, about 80 mg to about 95 mg, about 80 mg to about 90 mg, about 80 mg to about 85 mg, about 85 mg to about 95 mg, about 85 mg to about 90 mg, or about 90 mg to about 95 mg furosemide in its free acid form. In certain embodiments, a liquid pharmaceutical formulation described herein comprises a pharmaceutically acceptable salt of furosemide in an amount that provides about 40 mg to about 100 mg furosemide in its free acid form.

In certain embodiments, a liquid pharmaceutical formulation described herein comprises a pharmaceutically acceptable salt of furosemide in an amount that provides about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg furosemide in its free acid form. In certain embodiments, a liquid pharmaceutical formulation described herein comprises a pharmaceutically acceptable salt of furosemide in an amount that provides about 40 mg furosemide in its free acid form. In certain embodiments, a liquid pharmaceutical formulation described herein comprises a pharmaceutically acceptable salt of furosemide in an amount that provides about 80 mg furosemide in its free acid form.

In various embodiments, a liquid pharmaceutical formulation described herein comprises a pharmaceutically acceptable salt of furosemide in an amount that provides about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) furosemide in its free acid form. In certain embodiments, a liquid pharmaceutical formulation described herein comprises a pharmaceutically acceptable salt of furosemide in an amount that provides about 5% (w/w) to about 10% (w/w) furosemide in its free acid form.

In certain embodiments, a liquid pharmaceutical formulation described herein comprises a pharmaceutically acceptable salt of furosemide in an amount that provides about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) furosemide in its free acid form.

In certain embodiments, a liquid pharmaceutical formulation described herein comprises a pharmaceutically acceptable salt of furosemide in an amount that provides about 5% (w/w) furosemide. In certain embodiments, a liquid pharmaceutical formulation described herein comprises a pharmaceutically acceptable salt of furosemide in an amount that provides about 6% (w/w) furosemide in its free acid form. In certain embodiments, a liquid pharmaceutical formulation described herein comprises a pharmaceutically acceptable salt of furosemide in an amount that provides about 7% (w/w) furosemide in its free acid form. In certain embodiments, a liquid pharmaceutical formulation described herein comprises a pharmaceutically acceptable salt of furosemide in an amount that provides about 8% (w/w) furosemide in its free acid form. In certain embodiments, a liquid pharmaceutical formulation described herein comprises a pharmaceutically acceptable salt of furosemide in an amount that provides about 9% (w/w) furosemide in its free acid form. In certain embodiments, a liquid pharmaceutical formulation described herein comprises a pharmaceutically acceptable salt of furosemide in an amount that provides about 10% (w/w) furosemide in its free acid form.

In certain embodiments, a liquid pharmaceutical formulation described herein comprises a pharmaceutically acceptable salt of furosemide in an amount that provides a concentration of furosemide in its free acid form of 1 mg/mL to about 250 mg/mL, about 1 mg/ml to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL, about 1 mg/mL to about mg/mL mg, about 40 mg/mL to about 150 mg/mL, about 80 mg/mL to about 150 mg/mL, 1 mg/mL to about 250 mg/mL, about 1 mg/mL to about 200 mg/mL, about 40 mg/ml to about 250 mg/mL, about 40 mg/mL to about 200 mg/mL, about 80 mg/mL to about 250 mg/mL, about 80 mg/mL to about 200 mg/mL, about 50 mg/mL to about 250 mg/mL, about 60 mg/mL to about 250 mg/mL, about 70 mg/mL to about 250 mg/mL, about 80 mg/mL to about 250 mg/mL, about 90 mg/mL to about 250 mg/mL, about 100 mg/mL to about 250 mg/mL, about 120 mg/mL to about 250 mg/mL, about 140 mg/mL to about 250 mg/mL, about 160 mg/mL to about 250 mg/mL, about 180 mg/mL to about 250 mg/mL, about 200 mg/mL to about 250 mg/mL, about 50 mg/mL to about 200 mg/mL, about 50 mg/mL to about 180 mg/mL, about 50 mg/mL to about 160 mg/mL, about 50 mg/mL to about 140 mg/mL, about 50 mg/mL to about 120 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 200 mg/mL, about 60 mg/mL to about 180 mg/mL, about 60 mg/mL to about 160 mg/mL, about 60 mg/mL to about 140 mg/mL, about 60 mg/mL to about 120 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 200 mg/mL, about 70 mg/mL to about 180 mg/mL, about 70 mg/mL to about 160 mg/mL, about 70 mg/mL to about 140 mg/mL, about 70 mg/mL to about 120 mg/mL, about 70 mg/mL to about 100 mg/mL, about 70 mg/mL to about 90 mg/mL, about 70 mg/mL to about 80 mg/mL, about 80 mg/mL to about 200 mg/mL, about 80 mg/mL to about 180 mg/mL, about 80 mg/mL to about 160 mg/mL, about 80 mg/mL to about 140 mg/mL, about 80 mg/mL to about 120 mg/mL, about 80 mg/mL to about 100 mg/mL, about 80 mg/mL to about 90 mg/mL, about 90 mg/mL to about 200 mg/mL, about 90 mg/mL to about 180 mg/mL, about 90 mg/mL to about 160 mg/mL, about 90 mg/ml to about 140 mg/mL, about 90 mg/mL to about 120 mg/mL, about 90 mg/mL to about 100 mg/mL, about 100 mg/mL to about 200 mg/mL, about 100 mg/mL to about 180 mg/mL, about 100 mg/mL to about 160 mg/mL, about 100 mg/mL to about 140 mg/mL, about 100 mg/mL to about 120 mg/mL, about 120 mg/mL to about 200 mg/mL, about 120 mg/mL to about 180 mg/mL, about 120 mg/mL to about 160 mg/mL, about 120 mg/mL to about 140 mg/mL, about 140 mg/mL to about 200 mg/mL, about 140 mg/mL to about 180 mg/mL, about 140 mg/mL to about 160 mg/mL, about 160 mg/mL to about 200 mg/mL, about 160 mg/mL to about 180 mg/mL, or about 180 mg/mL to about 200 mg/mL.

In certain embodiments, a liquid pharmaceutical formulation described herein comprises a pharmaceutically acceptable salt of furosemide in an amount that provides a concentration of furosemide in its free acid form of 1 mg/mL to about 250 mg/mL, about 1 mg/mL to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL, about 1 mg/mL to about mg/mL mg, about 40 mg/mL to about 150 mg/mL, about 80 mg/mL to about 150 mg/mL, 1 mg/mL to about 250 mg/mL, about 1 mg/mL to about 200 mg/mL, about 40 mg/mL to about 250 mg/mL, about 40 mg/mL to about 200 mg/mL, about 80 mg/mL to about 250 mg/mL, about 80 mg/mL to about 200 mg/mL.

In certain embodiments, a liquid pharmaceutical formulation described herein comprises a pharmaceutically acceptable salt of furosemide in an amount that provides a concentration of furosemide in its free acid form of about 40 mg/mL to about 200 mg/mL. In certain embodiments, a liquid pharmaceutical formulation described herein comprises a pharmaceutically acceptable salt of furosemide in an amount that provides a concentration of furosemide in its free acid form of about 80 mg/mL to about 150 mg/mL.

In certain embodiments, a liquid pharmaceutical formulation described herein comprises a pharmaceutically acceptable salt of furosemide in an amount that provides a concentration of furosemide in its free acid form of about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, or about 250 mg/mL. In certain embodiments, a liquid pharmaceutical formulation described herein comprises a pharmaceutically acceptable salt of furosemide in an amount that provides a concentration of furosemide in its free acid form of about 80 mg/mL. In certain embodiments, a liquid pharmaceutical formulation described herein comprises a pharmaceutically acceptable salt of furosemide in an amount that provides a concentration of furosemide in its free acid form of about 100 mg/mL. In certain embodiments, a liquid pharmaceutical formulation described herein comprises a pharmaceutically acceptable salt of furosemide in an amount that provides a concentration of furosemide in its free acid form of about 120 mg/mL.

### Solubilizing Agents

In various embodiments, the liquid pharmaceutical formulation described herein may further comprise a solubilizing agent.

In an embodiment, the solubilizer is selected from the group consisting of a polysorbate, benzyl alcohol, polyethylene glycol, polyvinylpyrrolidone, N-methyl-2-pyrrolidone, ethanol, propylene glycol, and glycerin, or a combination thereof. In an embodiment, the solubilizer is selected from the group consisting of a polysorbate, benzyl alcohol, polyethylene glycol, polyvinylpyrrolidone, N-methyl-2-pyrrolidone, ethanol, propylene glycol, and glycerin, or a combination thereof.

In another embodiment, the solubilizer is benzyl alcohol. In yet another embodiment, the solubilizer is polyvinylpyrrolidone. In still embodiment, the solubilizer is N-methyl-2-pyrrolidone.

In various embodiments, a liquid pharmaceutical formulation described herein can comprise about 1 mg to about 50 mg, about 2 mg to about 50 mg, about 5 mg to about 50 mg, about 10 mg to about 50 mg, about 15 mg to about 50 mg, about 20 mg to about 50 mg, about 25 mg to about 50 mg, about 30 mg to about 50 mg, about 35 mg to about 50 mg, about 40 mg to about 50 mg, about 45 mg to about 50 mg, about 1 mg to about 45 mg, about 1 mg to about 40 mg, about 1 mg to about 35 mg, about 1 mg to about 30 mg, about 1 mg to about 25 mg, about 1 mg to about 20 mg, about 1 mg to about 15 mg, about 1 mg to about 10 mg, about 1 mg to about 5 mg, about 1 mg to about 2 mg, about 2 mg to about 45 mg, about 2 mg to about 40 mg, about 2 mg to about 35 mg, about 2 mg to about 30 mg, about 2 mg to about 25 mg, about 2 mg to about 20 mg, about 2 mg to about 15 mg, about 2 mg to about 10 mg, about 2 mg to about 5 mg, about 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 15 mg, about 5 mg to about 10 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 45 mg, about 20 mg to about 40 mg, about 20 mg to about 35 mg, about 20 mg to about 30 mg, about 20 mg to about 25 mg, about 25 mg to about 45 mg, about 25 mg to about 40 mg, about 25 mg to about 35 mg, about 25 mg to about 30 mg, about 30 mg to about 45 mg, about 30 mg to about 40 mg, about 30 mg to about 35 mg, about 35 mg to about 45 mg, about 35 mg to about 40 mg, or about 40 mg to about 45 mg of the solubilizing agent. In certain embodiments, a liquid pharmaceutical formulation described herein can comprise about 1 mg to about 50 mg of the solubilizing agent. In certain embodiments, a liquid pharmaceutical formulation described herein can comprise about 15 mg to about 50 mg of the solubilizing agent.

In various embodiments, a liquid pharmaceutical formulation described herein can comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg of the solubilizing agent.

In certain embodiments, a liquid pharmaceutical formulation described herein can comprise about 1 mg of the solubilizing agent. In certain embodiments, a liquid pharmaceutical formulation described herein can comprise about 2 mg of the solubilizing agent. In certain embodiments, a liquid pharmaceutical formulation described herein can comprise about 5 mg of the solubilizing agent. In certain embodiments, a liquid pharmaceutical formulation described herein can comprise about 10 mg of the solubilizing agent. In certain embodiments, a liquid pharmaceutical formulation described herein can comprise about 15 mg of the solubilizing agent. In certain embodiments, a liquid pharmaceutical formulation described herein can comprise about 20 mg of the solubilizing agent. In certain embodiments, a liquid pharmaceutical formulation described herein can comprise about 25 mg of the solubilizing agent. In certain embodiments, a liquid pharmaceutical formulation described herein can comprise about 30 mg of the solubilizing agent. In certain embodiments, a liquid pharmaceutical formulation described herein can comprise about 35 mg of the solubilizing agent. In certain embodiments, a liquid pharmaceutical formulation described herein can comprise about 40 mg of the solubilizing agent. In certain embodiments, a liquid pharmaceutical formulation described herein can comprise about 45 mg of the solubilizing agent. In certain embodiments, a liquid pharmaceutical formulation described herein can comprise about 50 mg of the solubilizing agent.

In various embodiments, a liquid pharmaceutical formulation described herein can comprise about 0.5% (w/w) to about 5% (w/w), about 1% (w/w) to about 5% (w/w), about 2% (w/w) to about 5% (w/w), about 3% (w/w) to about 5% (w/w), about 4% (w/w) to about 5% (w/w), about 1% (w/w) to about 4% (w/w), about 1% (w/w) to about 3% (w/w), about 1% (w/w) to about 2% (w/w), about 2% (w/w) to about 4% (w/w), about 2% (w/w) to about 3% (w/w), or about 3% (w/w) to about 4% (w/w) of the solubilizing agent.

In various embodiments, a liquid pharmaceutical formulation described herein can comprise about 0.5% (w/w) to about 5% (w/w) of the solubilizing agent.

In various embodiments, a liquid pharmaceutical formulation described herein can comprise about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.2% (w/w), about 2.4% (w/w), about 2.6% (w/w), about 2.8% (w/w), about 3% (w/w), about 3.5% (w/w), about 4% (w/w), about 4.5% (w/w), or about 5% (w/w) of the solubilizing agent.

In certain embodiments, a liquid pharmaceutical formulation described herein can comprise about 1% (w/w) of the solubilizing agent. In certain embodiments, a liquid pharmaceutical formulation described herein can comprise about 2% (w/w) of the solubilizing agent. In certain embodiments, a liquid pharmaceutical formulation described herein can comprise about 3% (w/w) of the solubilizing agent. In certain embodiments, a liquid pharmaceutical formulation described herein can comprise about 4% (w/w) of the solubilizing agent. In certain embodiments, a liquid pharmaceutical formulation described herein can comprise about 5% (w/w) of the solubilizing agent.

In various embodiments, the concentration of the solubilizing agent in a liquid pharmaceutical formulation described herein is about 15 mg/mL to about 50 mg/mL, about 20 mg/mL to about 50 mg/mL, about 25 mg/mL to about 50 mg/mL, about 30 mg/mL to about 50 mg/mL, about 35 mg/mL to about 50 mg/mL, about 40 mg/mL to about 50 mg/mL, about 45 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 45 mg/mL, about 30 mg/mL to about 40 mg/mL, about 30 mg/mL to about 35 mg/mL, about 35 mg/mL to about 45 mg/mL, about 35 mg/mL to about 40 mg/mL, or about 40 mg/mL to about 45 mg/mL.

In various embodiments, the concentration of the solubilizing agent in a liquid pharmaceutical formulation described herein is about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL.

Examples of solubilizing agents suitable for use in the liquid pharmaceutical formulations described herein include, but are not limited to, benzyl alcohol, a polysorbate (e.g., polysorbate 20 (e.g., Tween® 20) or polysorbate 80 (e.g., Tween® 80), sodium stearate, 4-(5-dodecyl)benzenesulfonate, docusate (dioctyl sodium sulfosuccinate), alkyl ether phosphates, sodium lauryl sulfate, polyethylene glycol ethers, polyoxyethylene 15 hydroxy stearate (e.g., Macrogol 15 hydroxy stearate, Solutol HS15®), polyoxyethylene castor oil derivatives (e.g., Cremophor® EL, ELP, RH 40), polyoxyethylene stearates (e.g., Myrj®), sorbitan fatty acid esters (e.g., Span®), polyoxyethylene alkyl ethers (e.g., Brij®), and polyoxyethylene nonylphenol ether (e.g., Nonoxynol®).

### Pharmaceutically Acceptable Buffers

In various embodiments, a liquid pharmaceutical formulation described herein may further comprise a pharmaceutically acceptable buffer selected from the group consisting of ammonium acetate, sodium bicarbonate, arginine, and glycine. In various embodiments, a liquid pharmaceutical formulation described herein may further comprise a pharmaceutically acceptable buffer selected from the group consisting of ammonium acetate, sodium bicarbonate, arginine, sodium tetraborate, potassium tetraborate, and glycine.

In an embodiment, the pharmaceutically acceptable buffer is an arginine buffer. In another embodiment, the pharmaceutically acceptable buffer is arginine HCl. In an embodiment, the pharmaceutically acceptable buffer is an arginine buffer or a potassium tetraborate buffer. In another embodiment, pharmaceutically acceptable buffer is sodium tetraborate. In another embodiment, pharmaceutically acceptable buffer is potassium tetraborate.

In various embodiments, a liquid pharmaceutical formulation described herein can comprise about 4 mg to about 16 mg, about 5 mg to about 16 mg, about 6 mg to about 16 mg, about 7 mg to about 16 mg, about 8 mg to about 16 mg, about 9 mg to about 16 mg, about 10 mg to about 16 mg, about 12 mg to about 16 mg, about 14 mg to about 16 mg, about 4 mg to about 14 mg, about 4 mg to about 12 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 14 mg, about 5 mg to about 12 mg, about 5 mg to about 10 mg, about 5 mg to about 9 mg, about 5 mg to about 8 mg, about 5 mg to about 7 mg, about 5 mg to about 6 mg, about 6 mg to about 14 mg, about 6 mg to about 12 mg, about 6 mg to about 10 mg, about 6 mg to about 9 mg, about 6 mg to about 8 mg, about 6 mg to about 7 mg, about 7 mg to about 14 mg, about 7 mg to about 12 mg, about 7 mg to about 10 mg, about 7 mg to about 9 mg, about 7 mg to about 8 mg, about 8 mg to about 14 mg, about 8 mg to about 12 mg, about 8 mg to about 10 mg, about 8 mg to about 9 mg, about 9 mg to about 14 mg, about 9 mg to about 12 mg, about 9 mg to about 10 mg, about 10 mg to about 14 mg, about 10 mg to about 12 mg, or about 12 mg to about 14 mg of the pharmaceutically acceptable buffer.

In certain embodiments, a liquid pharmaceutical formulation described herein can comprise about 4 mg to about 16 mg of the pharmaceutically acceptable buffer.

In various embodiments, a liquid pharmaceutical formulation described herein can comprise about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.5 mg, about 13 mg, about 13.5 mg, about 14 mg, about 14.5 mg, about 15 mg, about 15.5 mg, or about 16 mg of the pharmaceutically acceptable buffer. In certain embodiments, a liquid pharmaceutical formulation described herein can comprise about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, or about 10 mg of the pharmaceutically acceptable buffer.

In various embodiments, a liquid pharmaceutical formulation described herein comprises about 0.1% (w/w) to about 1.5% (w/w), 0.3% (w/w) to about 1.5% (w/w), about 0.5% (w/w) to about 1.5% (w/w), about 0.7% (w/w) to about 1.5% (w/w), about 0.9% (w/w) to about 1.5% (w/w), about 1.1% (w/w) to about 1.5% (w/w), about 1.3% (w/w) to about 1.5% (w/w), about 0.1% (w/w) to about 1.3% (w/w), about 0.1% (w/w) to about 1.1% (w/w), about 0.1% (w/w) to about 0.9% (w/w), about 0.1% (w/w) to about 0.7% (w/w), about 0.1% (w/w) to about 0.5% (w/w), about 0.1% (w/w) to about 0.3% (w/w), about 0.3% (w/w) to about 1.3% (w/w), about 0.3% (w/w) to about 1.1% (w/w), about 0.3% (w/w) to about 0.9% (w/w), about 0.3% (w/w) to about 0.7% (w/w), about 0.3% (w/w) to about 0.5% (w/w), about 0.5% (w/w) to about 1.3% (w/w), about 0.5% (w/w) to about 1.1% (w/w), about 0.5% (w/w) to about 0.9% (w/w), about 0.5% (w/w) to about 0.7% (w/w), about 0.7% (w/w) to about 1.3% (w/w), about 0.7% (w/w) to about 1.1% (w/w), about 0.7% (w/w) to about 0.9% (w/w), about 0.9% (w/w) to about 1.3% (w/w), about 0.9% (w/w) to about 1.1% (w/w), or about 1.1% (w/w) to about 1.3% (w/w) of the pharmaceutically acceptable buffer. In various embodiments, a liquid pharmaceutical formulation described herein comprises about 0.1% (w/w) to about 1.5% (w/w) of the pharmaceutically acceptable buffer.

In various embodiments, a liquid pharmaceutical formulation described herein comprises about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.1% (w/w), about 1.2% (w/w), about 1.3% (w/w), about 1.4% (w/w), or about 1.5% (w/w) of the pharmaceutically acceptable buffer. In various embodiments, a liquid pharmaceutical formulation described herein comprises about 0.5% (w/w) of the pharmaceutically acceptable buffer. In various embodiments, a liquid pharmaceutical formulation described herein comprises about 0.6% (w/w) of the pharmaceutically acceptable buffer. In various embodiments, a liquid pharmaceutical formulation described herein comprises about 0.7% (w/w) of the pharmaceutically acceptable buffer. In various embodiments, a liquid pharmaceutical formulation described herein comprises about 0.8% (w/w) of the pharmaceutically acceptable buffer. In various embodiments, a liquid pharmaceutical formulation described herein comprises about 0.9% (w/w) of the pharmaceutically acceptable buffer.

In various embodiments, the concentration of the pharmaceutically acceptable buffer in a liquid pharmaceutical formulation described herein is about 10 mM to 100 mM, 25 mM to 75 mM, about 50 mM to about 150 mM, about 60 mM to about 150 mM, about 70 mM to about 150 mM, about 80 mM to about 150 mM, about 90 mM to about 150 mM, about 100 mM to about 150 mM, about 110 mM to about 150 mM, about 120 mM to about 150 mM, about 130 mM to about 150 mM, about 140 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 140 mM, about 90 mM to about 130 mM, about 90 mM to about 120 mM, about 90 mM to about 110 mM, about 90 mM to about 100 mM, about 100 mM to about 140 mM, about 100 mM to about 130 mM, about 100 mM to about 120 mM, about 100 mM to about 110 mM, about 110 mM to about 140 mM, about 110 mM to about 130 mM, about 110 mM to about 120 mM, about 120 mM to about 140 mM, about 130 mM to about 140 mM, or about 130 mM to about 140 mM.

In an embodiment, the concentration of pharmaceutically acceptable buffer in the liquid pharmaceutical formulation is from about 10 mM to 100 mM. In another embodiment, the concentration of pharmaceutically acceptable buffer in the liquid pharmaceutical formulation is from about 25 mM to 75 mM.

In various embodiments, the concentration of the pharmaceutically acceptable buffer in a liquid pharmaceutical formulation described herein is about 50 mM, about 60 mM, about 70 mM, about 80 mM, about 90 mM, about 100 mM, about 110 mM, about 120 mM, about 130 mM, about 140 mM, or about 150 mM.

In certain embodiments, the concentration of the pharmaceutically acceptable buffer in a liquid pharmaceutical formulation described herein is about 50 mM. In certain embodiments, the concentration of the pharmaceutically acceptable buffer in a liquid pharmaceutical formulation described herein is about 60 mM. In certain embodiments, the concentration of the pharmaceutically acceptable buffer in a liquid pharmaceutical formulation described herein is about 70 mM. In certain embodiments, the concentration of the pharmaceutically acceptable buffer in a liquid pharmaceutical formulation described herein is about 80 mM.

In various embodiments, the concentration of the pharmaceutically acceptable buffer in a liquid pharmaceutical formulation described herein is about 5 mg/mL to about 15 mg/mL, about 6 mg/mL to about 15 mg/mL, about 7 mg/mL to about 15 mg/mL, about 8 mg/mL to about 15 mg/mL, about 9 mg/mL to about 15 mg/mL, about 10 mg/mL to about 15 mg/mL, about 11 mg/mL to about 15 mg/mL, about 12 mg/mL to about 15 mg/mL, about 13 mg/mL to about 15 mg/mL, about 14 mg/mL to about 15 mg/mL, about 5 mg/ml to about 14 mg/mL, about 5 mg/mL to about 13 mg/mL, about 5 mg/mL to about 12 mg/mL, about 5 mg/mL to about 11 mg/mL, about 5 mg/mL to about 10 mg/mL, about 5 mg/mL to about 9 mg/mL, about 5 mg/mL to about 8 mg/mL, about 5 mg/mL to about 7 mg/mL, about 5 mg/mL to about 6 mg/mL, about 6 mg/mL to about 14 mg/mL, about 6 mg/mL to about 13 mg/mL, about 6 mg/mL to about 12 mg/mL, about 6 mg/mL to about 11 mg/mL, about 6 mg/mL to about 10 mg/mL, about 6 mg/mL to about 9 mg/mL, about 6 mg/mL to about 8 mg/mL, about 6 mg/mL to about 7 mg/mL, about 7 mg/mL to about 14 mg/mL, about 7 mg/mL to about 13 mg/mL, about 7 mg/mL to about 12 mg/mL, about 7 mg/mL to about 11 mg/mL, about 7 mg/mL to about 10 mg/mL, about 7 mg/mL to about 9 mg/mL, about 7 mg/mL to about 8 mg/mL, about 8 mg/mL to about 14 mg/mL, about 8 mg/mL to about 13 mg/mL, about 8 mg/mL to about 12 mg/mL, about 8 mg/mL to about 11 mg/mL, about 8 mg/mL to about 10 mg/mL, about 8 mg/mL to about 9 mg/mL, about 9 mg/mL to about 14 mg/mL, about 9 mg/mL to about 13 mg/mL, about 9 mg/mL to about 12 mg/mL, about 9 mg/mL to about 11 mg/mL, about 9 mg/mL to about 10 mg/mL, about 10 mg/mL to about 14 mg/mL, about 10 mg/mL to about 13 mg/mL, about 10 mg/mL to about 12 mg/mL, about 10 mg/mL to about 11 mg/mL, about 11 mg/mL to about 14 mg/mL, about 11 mg/mL to about 13 mg/mL, about 11 mg/mL to about 12 mg/mL, about 12 mg/mL to about 14 mg/mL, about 12 mg/mL to about 13 mg/mL, or about 13 mg/mL to about 14 mg/mL. In certain embodiments, the concentration of the pharmaceutically acceptable buffer in a liquid pharmaceutical formulation described herein is about 5 mg/mL to about 15 mg/mL.

### pH Adjusters

In various embodiments, the liquid pharmaceutical formulation described herein further comprises a pharmaceutically acceptable pH adjuster.

Exemplary pharmaceutically acceptable pH adjusters include, but are not limited to, one or more of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, and trolamine.

In certain embodiments, the pharmaceutically acceptable pH adjuster is selected from the group consisting of potassium hydroxide, sodium hydroxide, hydrochloric acid, and combinations thereof. In certain embodiments, the pharmaceutically acceptable pH adjuster is hydrochloric acid and sodium hydroxide. In certain embodiments, the pharmaceutically acceptable pH adjuster is sodium hydroxide. In certain embodiments, the pharmaceutically acceptable pH adjuster is potassium hydroxide. In certain embodiments, the pharmaceutically acceptable pH adjuster is hydrochloric acid.

### pH of a Liquid Pharmaceutical Formulation

In various embodiments, the pH of a liquid pharmaceutical formulation described herein can be from about 5.5 to about 8.5, from about 6 to about 8.5, from about 6.5 to about 8.5, from about 7 to about 8.5, from about 7.5 to about 8.5, from about 8 to about 8.5, from about 5.5 to about 8, from about 5.5 to about 7.5, from about 5.5 to about 7, from about 5.5 to about 6.5, from about 5.5 to about 6, from about 6 to about 8, from about 6 to about 7.5, from about 6 to about 7, from about 6 to about 6.5, from about 6.5 to about 8, from about 6.5 to about 7.5, from about 6.5 to about 7, from about 7 to about 8, from about 7 to about 7.5, or from about 7.5 to about 8. In certain embodiments, the pH of a liquid pharmaceutical formulation described herein is from about 6.5 to about 8.5. In certain embodiments, the pH of a liquid pharmaceutical formulation described herein is from about 7 to about 8. In certain embodiments, the pH of a liquid pharmaceutical formulation described herein is from about 7.5 to about 8.5.

In certain embodiments, the pH of a liquid pharmaceutical formulation described herein can be from about 7 to about 8.5, from about 7.1 to about 8.5, from about 7.3 to about 8.5, from about 7.5 to about 8.5, from about 7.7 to about 8.5, from about 7.9 to about 8.5, from about 8.1 to about 8.5, from about 8.3 to about 8.5, about 7 to about 8.3, about 7 to about 8.1, about 7 to about 7.9, about 7 to about 7.7, about 7 to about 7.5, about 7 to about 7.3, about 7 to about 7.1, about 7.1 to about 8.3, about 7.1 to about 8.1, about 7.1 to about 7.9, about 7.1 to about 7.7, about 7.1 to about 7.5, about 7.1 to about 7.3, about 7.3 to about 8.3, about 7.3 to about 8.1, about 7.3 to about 7.9, about 7.3 to about 7.7, about 7.3 to about 7.5, about 7.5 to about 8.3, about 7.5 to about 8.1, about 7.5 to about 7.9, about 7.5 to about 7.7, about 7.7 to about 8.3, about 7.7 to about 8.1, about 7.7 to about 7.9, about 7.9 to about 8.3, about 7.9 to about 8.1, or about 8.1 to about 8.3.

In certain embodiments, the pH of a liquid pharmaceutical formulation described herein can be about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, or about 8.5. In certain embodiments, the pH of a liquid pharmaceutical formulation described herein can be about 7.4. In certain embodiments, the pH of a liquid pharmaceutical formulation described herein can be about 7.5. In certain embodiments, the pH of a liquid pharmaceutical formulation described herein can be about 7.6. In certain embodiments, the pH of a liquid pharmaceutical formulation described herein can be about 7.7. In certain embodiments, the pH of a liquid pharmaceutical formulation described herein can be about 7.8. In certain embodiments, the pH of a liquid pharmaceutical formulation described herein can be about 7.9. In certain embodiments, the pH of a liquid pharmaceutical formulation described herein can be about 8.0.

In certain embodiments, the pH of a liquid pharmaceutical formulation described herein can be 5.5±0.3, 5.6±0.3, 5.7±0.3, 5.8±0.3, 5.9±0.3, 6±0.3, 6.1±0.3, 6.2±0.3, 6.3±0.3, 6.4±0.3, 6.5±0.3, 6.6±0.3, 6.7±0.3, 6.8±0.3, 6.9±0.3, 7±0.3, 7.1±0.3, 7.2±0.3, 7.3±0.3, 7.4±0.3, 7.5±0.3, 7.6±0.3, 7.7±0.3, 7.8±0.3, 7.9±0.3, 8±0.3, 8.1±0.3, 8.2±0.3, 8.3±0.3, 8.4±0.3, or 8.5±0.3. In certain embodiments, the pH of a liquid pharmaceutical formulation described herein can be 7.4±0.3. In certain embodiments, the pH of a liquid pharmaceutical formulation described herein can be 7.5±0.3. In certain embodiments, the pH of a liquid pharmaceutical formulation described herein can be 7.6±0.3. In certain embodiments, the pH of a liquid pharmaceutical formulation described herein can be 7.7±0.3. In certain embodiments, the pH of a liquid pharmaceutical formulation described herein can be 7.8±0.3. In certain embodiments, the pH of a liquid pharmaceutical formulation described herein can be 7.9±0.3. In certain embodiments, the pH of a liquid pharmaceutical formulation described herein can be 8.0±0.3.

### Additional Pharmaceutically Acceptable Excipients

### (1) Osmolarity and Osmolality

In various embodiments, a liquid pharmaceutical formulation described herein can further comprise an osmolarity or an osmolality adjuster.

In various embodiments, the osmolarity or the osmolality adjuster is selected from the group comprising sodium chloride, potassium chloride, isosorbide, mannitol, xylitol, and combinations thereof. In certain embodiments, the osmolarity or the osmolality adjuster is sodium chloride, potassium chloride, or a combination thereof. In certain embodiments, the osmolarity or the osmolality adjuster is sodium chloride. In certain embodiments, the osmolarity or the osmolality adjuster is potassium chloride.

In various embodiments, a liquid pharmaceutical formulation described herein has an osmolarity in the range of from about 100 mOsm/kg to about 1600 mOsm/kg, from about 200 mOsm/kg to about 1600 mOsm/kg, from about 300 mOsm/kg to about 1600 mOsm/kg, from about 400 mOsm/kg to about 1600 mOsm/kg, from about 600 mOsm/kg to about 1600 mOsm/kg, from about 800 mOsm/kg to about 1600 mOsm/kg, from about 1200 mOsm/kg to about 1600 mOsm/kg, from about 100 mOsm/kg to about 1200 mOsm/kg, from about 100 mOsm/kg to about 800 mOsm/kg, from about 100 mOsm/kg to about 600 mOsm/kg, from about 100 mOsm/kg to about 400 mOsm/kg, from about 100 mOsm/kg to about 300 mOsm/kg, from about 100 mOsm/kg to about 200 mOsm/kg, from about 200 mOsm/kg to about 1200 mOsm/kg, from about 200 mOsm/kg to about 800 mOsm/kg, from about 200 mOsm/kg to about 600 mOsm/kg, from about 200 mOsm/kg to about 400 mOsm/kg, from about 200 mOsm/kg to about 300 mOsm/kg, from about 300 mOsm/kg to about 1200 mOsm/kg, from about 300 mOsm/kg to about 800 mOsm/kg, from about 300 mOsm/kg to about 600 mOsm/kg, from about 300 mOsm/kg to about 400 mOsm/kg, from about 400 mOsm/kg to about 1200 mOsm/kg, from about 400 mOsm/kg to about 800 mOsm/kg, from about 400 mOsm/kg to about 600 mOsm/kg, from about 600 mOsm/kg to about 1200 mOsm/kg, from about 600 mOsm/kg to about 800 mOsm/kg, or from about 800 mOsm/kg to about 1200 mOsm/kg. In certain embodiments, a liquid pharmaceutical formulation described herein has an osmolarity in the range of from about 200 mOsm/kg to about 400 mOsm/kg. In certain embodiments, a liquid pharmaceutical formulation described herein has an osmolarity in the range of from about 300 mOsm/kg to about 600 mOsm/kg. In certain embodiments, a liquid pharmaceutical formulation described herein has an osmolarity in the range of from about 300 mOsm/kg to about 450 mOsm/kg. In certain embodiments, a liquid pharmaceutical formulation described herein has an osmolarity in the range of about 100 mOsm/kg to about 1600 mOsm/kg. In certain embodiments, a liquid pharmaceutical formulation described herein has an osmolarity in the range of about 200 mOsm/kg to about 800 mOsm/kg. In certain embodiments, a liquid pharmaceutical formulation described herein has an osmolarity in the range of about 200 mOsm/kg to about 600 mOsm/kg. In certain embodiments, a liquid pharmaceutical formulation described herein has an osmolarity in the range of about 200 mOsm/kg to about 400 mOsm/kg. In certain embodiments, a liquid pharmaceutical formulation described herein has an osmolarity in the range of from about 275 mOsm/kg to about 400 mOsm/kg.

It should be understood that the above ranges and description of osmolarity are equally applicable to osmolality but with the units of “Osm/kg.”

### (2) Water

In various embodiments, a liquid pharmaceutical formulation described herein further comprises water. In certain embodiments, the water is water for injection.

In various embodiments, a liquid pharmaceutical formulation described herein can comprise about 0.1 mL to about 2 mL, about 0.2 mL to about 2 mL, about 0.3 mL to about 2 mL, about 0.4 mL to about 2 mL, about 0.5 mL to about 2 mL, about 0.6 mL to about 2 mL, about 0.7 mL to about 2 mL, about 0.8 mL to about 2 mL, about 0.9 mL to about 2 mL, about 1 mL to about 2 mL, about 1.5 mL to about 2 mL, about 0.1 mL to about 1.5 mL, about 0.1 mL to about 1 mL, about 0.1 mL to about 0.9 mL, about 0.1 mL to about 0.8 mL, about 0.1 mL to about 0.7 mL, about 0.1 mL to about 0.6 mL, about 0.1 mL to about 0.5 mL, about 0.1 mL to about 0.4 mL, about 0.1 mL to about 0.3 mL, about 0.1 mL to about 0.2 mL, about 0.2 mL to about 1.5 mL, about 0.2 mL to about 1 mL, about 0.2 mL to about 0.9 mL, about 0.2 mL to about 0.8 mL, about 0.2 mL to about 0.7 mL, about 0.2 mL to about 0.6 mL, about 0.2 mL to about 0.5 mL, about 0.2 mL to about 0.4 mL, about 0.2 mL to about 0.3 mL, about 0.3 mL to about 1.5 mL, about 0.3 mL to about 1 mL, about 0.3 mL to about 0.9 mL, about 0.3 mL to about 0.8 mL, about 0.3 mL to about 0.7 mL, about 0.3 mL to about 0.6 mL, about 0.3 mL to about 0.5 mL, about 0.3 mL to about 0.4 mL, about 0.4 mL to about 1.5 mL, about 0.4 mL to about 1 mL, about 0.4 mL to about 0.9 mL, about 0.4 mL to about 0.8 mL, about 0.4 mL to about 0.7 mL, about 0.4 mL to about 0.6 mL, about 0.4 mL to about 0.5 mL, about 0.5 mL to about 1.5 mL, about 0.5 mL to about 1 mL, about 0.5 mL to about 0.9 mL, about 0.5 mL to about 0.8 mL, about 0.5 mL to about 0.7 mL, about 0.5 mL to about 0.6 mL, about 0.6 mL to about 1.5 mL, about 0.6 mL to about 1 mL, about 0.6 mL to about 0.9 mL, about 0.6 mL to about 0.8 mL, about 0.6 mL to about 0.7 mL, about 0.7 mL to about 1.5 mL, about 0.7 mL to about 1 mL, about 0.7 mL to about 0.9 mL, about 0.7 mL to about 0.8 mL, about 0.8 mL to about 1.5 mL, about 0.8 mL to about 1 mL, about 0.8 mL to about 0.9 mL, about 0.9 mL to about 1.5 mL, about 0.9 mL to about 1 mL, or about 1 mL to about 1.5 mL water.

In various embodiments, the liquid pharmaceutical compositions described herein can comprise about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL water. In certain embodiments, the liquid pharmaceutical compositions described herein can comprise about 0.3 mL water. In certain embodiments, the liquid pharmaceutical compositions described herein can comprise about 0.4 mL water. In certain embodiments, the liquid pharmaceutical compositions described herein can comprise about 0.5 mL water. In certain embodiments, the liquid pharmaceutical compositions described herein can comprise about 0.6 mL water. In certain embodiments, the liquid pharmaceutical compositions described herein can comprise about 0.7 mL water. In certain embodiments, the liquid pharmaceutical compositions described herein can comprise about 0.8 mL water. In certain embodiments, the liquid pharmaceutical compositions described herein can comprise about 0.9 mL water. In certain embodiments, the liquid pharmaceutical compositions described herein can comprise about 1 mL water. In certain embodiments, the liquid pharmaceutical compositions described herein can comprise about 1.1 mL water. In certain embodiments, the liquid pharmaceutical compositions described herein can comprise about 1.2 mL water.

It should be understood that the volume of water added to the pharmaceutical formulation can be an amount to bring the total volume to one of the enumerated volumes of water herein, for example, to bring the total volume of the pharmaceutical formulation to about 0.1 mL, about 0.2 mL, about 0.3 mL, about 0.4 mL, 0.5 mL, 0.6 mL, 0.7 mL, 0.8 mL, 0.9 mL, or 1.0 mL.

In various embodiments, the liquid pharmaceutical compositions described herein can comprise about 0.1 g to about 2 g, about 0.2 g to about 2 g, about 0.3 g to about 2 g, about 0.4 g to about 2 g, about 0.5 g to about 2 g, about 0.6 g to about 2 g, about 0.7 g to about 2 g, about 0.8 g to about 2 g, about 0.9 g to about 2 g, about 1 g to about 2 g, about 1.5 g to about 2 g, about 0.1 g to about 1.5 g, about 0.1 g to about 1 g, about 0.1 g to about 0.9 g, about 0.1 g to about 0.8 g, about 0.1 g to about 0.7 g, about 0.1 g to about 0.6 g, about 0.1 g to about 0.5 g, about 0.1 g to about 0.4 g, about 0.1 g to about 0.3 g, about 0.1 g to about 0.2 g, about 0.2 g to about 1.5 g, about 0.2 g to about 1 g, about 0.2 g to about 0.9 g, about 0.2 g to about 0.8 g, about 0.2 g to about 0.7 g, about 0.2 g to about 0.6 g, about 0.2 g to about 0.5 g, about 0.2 g to about 0.4 g, about 0.2 g to about 0.3 g, about 0.3 g to about 1.5 g, about 0.3 g to about 1 g, about 0.3 g to about 0.9 g, about 0.3 g to about 0.8 g, about 0.3 g to about 0.7 g, about 0.3 g to about 0.6 g, about 0.3 g to about 0.5 g, about 0.3 g to about 0.4 g, about 0.4 g to about 1.5 g, about 0.4 g to about 1 g, about 0.4 g to about 0.9 g, about 0.4 g to about 0.8 g, about 0.4 g to about 0.7 g, about 0.4 g to about 0.6 g, about 0.4 g to about 0.5 g, about 0.5 g to about 1.5 g, about 0.5 g to about 1 g, about 0.5 g to about 0.9 g, about 0.5 g to about 0.8 g, about 0.5 g to about 0.7 g, about 0.5 g to about 0.6 g, about 0.6 g to about 1.5 g, about 0.6 g to about 1 g, about 0.6 g to about 0.9 g, about 0.6 g to about 0.8 g, about 0.6 g to about 0.7 g, about 0.7 g to about 1.5 g, about 0.7 g to about 1 g, about 0.7 g to about 0.9 g, about 0.7 g to about 0.8 g, about 0.8 g to about 1.5 g, about 0.8 g to about 1 g, about 0.8 g to about 0.9 g, about 0.9 g to about 1.5 g, about 0.9 g to about 1 g, or about 1 g to about 1.5 g water.

In various embodiments, the liquid pharmaceutical compositions described herein can comprise about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, or about 2 g water. In certain embodiments, the liquid pharmaceutical compositions described herein can comprise about 0.3 g water. In certain embodiments, the liquid pharmaceutical compositions described herein can comprise about 0.4 g water. In certain embodiments, the liquid pharmaceutical compositions described herein can comprise about 0.5 g water. In certain embodiments, the liquid pharmaceutical compositions described herein can comprise about 0.6 g water. In certain embodiments, the liquid pharmaceutical compositions described herein can comprise about 0.7 g water. In certain embodiments, the liquid pharmaceutical compositions described herein can comprise about 0.8 g water. In certain embodiments, the liquid pharmaceutical compositions described herein can comprise about 0.9 g water. In certain embodiments, the liquid pharmaceutical compositions described herein can comprise about 1 g water. In certain embodiments, the liquid pharmaceutical compositions described herein can comprise about 1.1 g water. In certain embodiments, the liquid pharmaceutical compositions described herein can comprise about 1.2 g water.

### (3) Other Excipients

In various embodiments, the liquid pharmaceutical formulations described herein may further comprise one or more additional pharmaceutically acceptable excipients.

In various embodiments, the one or more additional pharmaceutically acceptable excipients is selected from the group consisting of ethanol, glycerin, N-methyl-pyrrolidone (NMP), sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In certain embodiments, the one or more additional pharmaceutically acceptable excipients comprises ethanol. In certain embodiments, the one or more additional pharmaceutically acceptable excipients comprises glycerin. In certain embodiments, the one or more additional pharmaceutically acceptable excipients comprises N-methyl-pyrrolidone (NMP). In certain embodiments, the one or more additional pharmaceutically acceptable excipients comprises sodium carbonate. In certain embodiments, the one or more additional pharmaceutically acceptable excipients comprises mannitol. In certain embodiments, the one or more additional pharmaceutically acceptable excipients comprises lactose. In certain embodiments, the one or more additional pharmaceutically acceptable excipients comprises dextrose. In certain embodiments, the one or more additional pharmaceutically acceptable excipients comprises a polyethylene glycol (PEG). In certain embodiments, the one or more additional pharmaceutically acceptable excipients comprises propylene glycol. In certain embodiments, the one or more additional pharmaceutically acceptable excipients comprises a polysorbate (e.g., polyoxyethylene (20) sorbitan monolaurate, polyoxyethylene (20) sorbitan monopalmitate, polyoxyethylene (20) sorbitan monostearate, polyoxyethylene (20) sorbitan monooleate). In certain embodiments, the one or more additional pharmaceutically acceptable excipients comprises a polyvinylpyrrolidone (PVP). In certain embodiments, the one or more additional pharmaceutically acceptable excipients comprises a cyclodextrin (e.g., an a (alpha)-cyclodextrin, a β (beta)-cyclodextrin, and a γ (gamma)-cyclodextrin). In certain embodiments, the one or more additional pharmaceutically acceptable excipients comprises a cyclodextrin derivative (e.g., a sulfobutyl-ether-β-cyclodextrin (e.g., captisol), a hydroxypropyl-β-cyclodextrin (e.g., 2-hydroxypropyl-β-cyclodextrin), and a methylated β-cyclodextrin (e.g., a randomly methylated β-cyclodextrin)). In certain embodiments, the one or more additional pharmaceutically acceptable excipients comprises vitamin E. In certain embodiments, the one or more additional pharmaceutically acceptable excipients comprises one or more naturally occurring forms of vitamin E (e.g., alpha-, beta-, gamma-, and delta-tocopherol and alpha-, beta-, gamma-, and delta-tocotrienol). In certain embodiments, the one or more additional pharmaceutically acceptable excipients comprises vitamin E derivative (e.g., tocopheryl acetate, tocopheryl glucoside, tocopheryl phosphate).

### Formulation Volume

In various embodiments, the total volume of a liquid pharmaceutical formulation described herein can be about 0.1 mL to about 2 mL, about 0.2 mL to about 2 mL, about 0.3 mL to about 2 mL, about 0.4 mL to about 2 mL, about 0.5 mL to about 2 mL, about 0.6 mL to about 2 mL, about 0.7 mL to about 2 mL, about 0.8 mL to about 2 mL, about 0.9 mL to about 2 mL, about 1 mL to about 2 mL, about 1.5 mL to about 2 mL, about 0.1 mL to about 1.5 mL, about 0.1 mL to about 1 mL, about 0.1 mL to about 0.9 mL, about 0.1 mL to about 0.8 mL, about 0.1 mL to about 0.7 mL, about 0.1 mL to about 0.6 mL, about 0.1 mL to about 0.5 mL, about 0.1 ml to about 0.4 mL, about 0.1 mL to about 0.3 mL, about 0.1 mL to about 0.2 mL, about 0.2 mL to about 1.5 mL, about 0.2 mL to about 1 mL, about 0.2 mL to about 0.9 mL, about 0.2 mL to about 0.8 mL, about 0.2 mL to about 0.7 mL, about 0.2 mL to about 0.6 mL, about 0.2 mL to about 0.5 mL, about 0.2 mL to about 0.4 mL, about 0.2 mL to about 0.3 mL, about 0.3 mL to about 1.5 mL, about 0.3 mL to about 1 mL, about 0.3 mL to about 0.9 mL, about 0.3 mL to about 0.8 mL, about 0.3 mL to about 0.7 mL, about 0.3 mL to about 0.6 mL, about 0.3 mL to about 0.5 mL, about 0.3 mL to about 0.4 mL, about 0.4 mL to about 1.5 mL, about 0.4 mL to about 1 mL, about 0.4 mL to about 0.9 mL, about 0.4 mL to about 0.8 mL, about 0.4 mL to about 0.7 mL, about 0.4 mL to about 0.6 mL, about 0.4 mL to about 0.5 mL, about 0.5 mL to about 1.5 mL, about 0.5 mL to about 1 mL, about 0.5 mL to about 0.9 mL, about 0.5 mL to about 0.8 mL, about 0.5 mL to about 0.7 mL, about 0.5 mL to about 0.6 mL, about 0.6 mL to about 1.5 mL, about 0.6 mL to about 1 mL, about 0.6 mL to about 0.9 mL, about 0.6 mL to about 0.8 mL, about 0.6 mL to about 0.7 mL, about 0.7 mL to about 1.5 mL, about 0.7 mL to about 1 mL, about 0.7 mL to about 0.9 mL, about 0.7 mL to about 0.8 mL, about 0.8 mL to about 1.5 mL, about 0.8 mL to about 1 mL, about 0.8 mL to about 0.9 mL, about 0.9 mL to about 1.5 mL, about 0.9 mL to about 1 mL, or about 1 mL to about 1.5 mL.

In various embodiments, the total volume of a liquid pharmaceutical formulation described herein can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL. In certain embodiments, the total volume of a liquid pharmaceutical formulation described herein is about 0.3 mL. In certain embodiments, the total volume of a liquid pharmaceutical formulation described herein is about 0.4 mL. In certain embodiments, the total volume of a liquid pharmaceutical formulation described herein is about 0.5 mL. In certain embodiments, the total volume of a liquid pharmaceutical formulation described herein is about 0.6 mL. In certain embodiments, the total volume of a liquid pharmaceutical formulation described herein is about 0.7 mL. In certain embodiments, the total volume of a liquid pharmaceutical formulation described herein is about 0.8 mL. In certain embodiments, the total volume of a liquid pharmaceutical formulation described herein is about 0.9 mL. In certain embodiments, the total volume of a liquid pharmaceutical formulation described herein is about 1 mL. In certain embodiments, the total volume of a liquid pharmaceutical formulation described herein is about 1.1 mL. In certain embodiments, the total volume of a liquid pharmaceutical formulation described herein is about 1.2 mL.

### Exemplary Liquid Pharmaceutical Formulations

In an aspect, provided herein is a liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, and glycine.

In another aspect, provided herein is a liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, sodium tetraborate, potassium tetraborate, and glycine.

In an embodiment, the pharmaceutically acceptable buffer is an arginine buffer. In another embodiment, the pharmaceutically acceptable buffer is arginine HCl. In an embodiment, the pharmaceutically acceptable buffer is arginine or potassium. In another embodiment, pharmaceutically acceptable buffer is sodium tetraborate. In another embodiment, pharmaceutically acceptable buffer is potassium tetraborate.

In yet another embodiment, the concentration of pharmaceutically acceptable buffer in the liquid pharmaceutical formulation is from about 10 mM to 100 mM. In still another embodiment, the concentration of pharmaceutically acceptable buffer in the liquid pharmaceutical formulation is from about 25 mM to 75 mM. In an embodiment, the concentration of pharmaceutically acceptable buffer in the liquid pharmaceutical formulation is about 50 mM.

In another embodiment, the solubilizer is selected from the group consisting of a polysorbate, benzyl alcohol, polyethylene glycol, polyvinylpyrrolidone, N-methyl-2-pyrrolidone, ethanol, propylene glycol, and glycerin, or a combination thereof.

In yet another embodiment, the solubilizer is benzyl alcohol. In still another embodiment, the solubilizer is polyvinylpyrrolidone. In an embodiment, the solubilizer is N-methyl-2-pyrrolidone.

In an embodiment, the amount of solubilizer in the liquid pharmaceutical formulation is from 0.5% (w/w) to 5% (w/w). In another embodiment, the amount of solubilizer in the liquid pharmaceutical formulation is about 1% (w/w).

In yet another embodiment, the buffer is an arginine buffer and the solubilizer is benzyl alcohol, polyvinylpyrrolidone, N-methyl-2-pyrrolidone, or a combination thereof. In another embodiment, the buffer is an arginine buffer and the solubilizer is benzyl alcohol. In another embodiment, the buffer is sodium tetraborate and the solubilizer is benzyl alcohol. In another embodiment, the buffer is potassium tetraborate and the solubilizer is benzyl alcohol.

In another embodiment, the buffer is an arginine buffer and the solubilizer is a cyclodextrin. In an embodiment, the cyclodextrin is selected from the group consisting of β-cyclodextrin, hydroxypropyl-β-cyclodextrin, methylated β-cyclodextrin, and β-cyclodextrin sulfobutyl ether. In an embodiment, the cyclodextrin is hydroxypropyl-β-cyclodextrin.

In an embodiment, the liquid pharmaceutical formulation optionally further comprises (iv) a pH adjuster. In another embodiment, the pH adjuster is potassium hydroxide or sodium hydroxide.

In yet another embodiment, the concentration of furosemide in the liquid pharmaceutical formulation is from about 1 mg/mL to about 250 mg/mL. In still another embodiment, the concentration of furosemide in the liquid pharmaceutical formulation is from about 40 mg/mL to about 200 mg/mL. In an embodiment, the concentration of furosemide in the liquid pharmaceutical formulation is from about 80 mg/mL to about 150 mg/mL.

In another embodiment, the pH of the liquid pharmaceutical formulation is from about 6.5 to about 8.5. In yet another embodiment, the pH of the liquid pharmaceutical formulation is from about 7 to about 8.3.

In another aspect, provided herein is a unit liquid pharmaceutical formulation comprises:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, and glycine; and optionally
(iv) a pH adjuster that is potassium hydroxide or sodium hydroxide.

In another aspect, provided herein is a unit liquid pharmaceutical formulation comprises:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, a cyclodextrin, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, sodium tetraborate, potassium tetraborate, and glycine; and optionally
(iv) a pH adjuster that is potassium hydroxide or sodium hydroxide.

In an embodiment, the liquid pharmaceutical formulation comprises:
(i) from about 1 mg to about 250 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of arginine buffer; and optionally
(iv) a pH adjuster that is potassium hydroxide.

In an embodiment, the liquid pharmaceutical formulation comprises:
(i) from about 1 mg to about 250 mg of furosemide, or a pharmaceutically acceptable salt thereof;
(ii) from about 0.1% (w/w) to about 30% (w/w) of benzyl alcohol, a cyclodextrin, or a combination thereof;
(iii) from about 10 mM to 100 mM of arginine buffer; and optionally
(iv) a pH adjuster that is potassium hydroxide.

In an embodiment, the liquid pharmaceutical formulation comprises:
(i) from about 1 mg to about 250 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of sodium tetraborate; and optionally
(iv) a pH adjuster that is potassium hydroxide.

In an embodiment, the liquid pharmaceutical formulation comprises:
(i) from about 1 mg to about 250 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of potassium tetraborate; and optionally
(iv) a pH adjuster that is potassium hydroxide.

In another embodiment, the liquid pharmaceutical formulation comprises:
(i) from about 60 mg to about 90 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of arginine buffer; and optionally
(iv) a pH adjuster that is potassium hydroxide.

In another embodiment, the liquid pharmaceutical formulation comprises:
(i) from about 60 mg to about 90 mg of furosemide, or a pharmaceutically acceptable salt thereof;
(ii) from about 0.1% (w/w) to about 30% (w/w) of benzyl alcohol, potassium tetraborate, hydroxypropyl-β-cyclodextrin, or a combination thereof;
(iii) from about 10 mM to 100 mM of arginine buffer; and optionally
(iv) a pH adjuster that is potassium hydroxide.

In an embodiment, the liquid pharmaceutical formulation comprises 40 mg of furosemide. In another embodiment, the liquid pharmaceutical formulation comprises 80 mg of furosemide.

In an embodiment, the liquid pharmaceutical formulation comprises a first solubilizer and a second solubilizer. In another embodiment, the liquid pharmaceutical formulation comprises a first solubilizer.

### Methods of Treatment

In one aspect, a liquid pharmaceutical formulation described herein may be used for the treatment of a variety of conditions such as, but not limited to, congestion due to fluid overload, edema, and hypertension in a subject in need thereof. In various embodiments, the condition is selected from the group consisting of congestion due to fluid overload, edema, and hypertension, and combinations thereof. In certain embodiments, the condition is congestion due to fluid overload. In certain embodiments, the condition is edema. In certain embodiments, the condition is hypertension. In certain embodiments, the edema is associated with congestive heart failure, cirrhosis of the liver or renal disease. In certain embodiments, the renal disease is nephrotic syndrome. In an embodiment, the subject has chronic heart failure. In another embodiment, the subject has chronic kidney disease.

In certain embodiments, the subject is a human.

In an embodiment, the liquid pharmaceutical formulation is administered to the patient by subcutaneous injection. In another embodiment, the unit liquid pharmaceutical formulation is administered to the patient by subcutaneous injection. In yet another embodiment, the unit liquid pharmaceutical formulation is administered to the patient by a subcutaneous auto-injector.

In various embodiments, provided herein is a method of treating a condition selected from the group consisting of congestion due to fluid overload, edema, and hypertension, in a human in need thereof, administering to the human via subcutaneous injection about 1 mL or less of a liquid pharmaceutical formulation described herein.

In various embodiments, provided herein is a method of treating a condition selected from the group consisting of congestion due to fluid overload, edema, and hypertension, in a human in need thereof, administering to the human via subcutaneous injection an effective amount of furosemide in a total volume of about 1 mL or less of a liquid pharmaceutical formulation.

In another embodiment, the methods herein comprise administering to a subject in need thereof a liquid pharmaceutical formulation that comprises:
(i) from about 60 mg to about 90 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of arginine buffer; and optionally
(iv) a pH adjuster that is potassium hydroxide.

In another embodiment, the methods herein comprise administering to a subject in need thereof a liquid pharmaceutical formulation that comprises:
(i) from about 60 mg to about 90 mg of furosemide, or a pharmaceutically acceptable salt thereof;
(ii) from about 0.1% (w/w) to about 30% (w/w) of benzyl alcohol, a cyclodextrin, or a combination thereof;
(iii) from about 10 mM to 100 mM of arginine buffer; and optionally
(iv) a pH adjuster that is potassium hydroxide.

In another embodiment, the methods herein comprise administering to a subject in need thereof a liquid pharmaceutical formulation that comprises:
(i) from about 60 mg to about 90 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of sodium tetraborate; and optionally
(iv) a pH adjuster that is potassium hydroxide.

In another embodiment, the methods herein comprise administering to a subject in need thereof a liquid pharmaceutical formulation that comprises:
(i) from about 60 mg to about 90 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of potassium tetraborate; and optionally
(iv) a pH adjuster that is potassium hydroxide.

### Kits

In one aspect, provided herein are kits for the treatment or prevention of a variety of conditions described supra in a patient in need thereof. In certain embodiments, the condition, disease, or disorder is selected from the group consisting of congestion due to fluid overload, edema, and hypertension. In certain embodiments, the condition is congestion due to fluid overload. In certain embodiments, the condition is edema. In certain embodiments, the condition is hypertension. In certain embodiments, the edema is associated with congestive heart failure, cirrhosis of the liver or renal disease. In certain embodiments, the renal disease is nephrotic syndrome.

In various embodiments, a kit comprises a liquid pharmaceutical formulation described herein. In certain embodiments, the kit comprises one or more unit doses of a liquid pharmaceutical formulation described herein. In an embodiment, the kit comprises one, two, three or more units. In another embodiment, the kit further comprises an auto-injector. In certain embodiments, the kit further comprises a medical device. In certain embodiments, the kit further comprises instructions for treating a disease or disorder of the present disclosure.

A number of medical devices have been proposed to facilitate self-administration of a liquid pharmaceutical formulation. The device may include a reservoir containing, for example, pre-loaded with, a liquid pharmaceutical formulation described herein to be administered. A type of device useful for the intramuscular or subcutaneous delivery of pharmaceutical formulations is an autoinjector. In various embodiments, the kit comprises an autoinjector. In various embodiments, the kit comprises a patient-controlled injector.

Accordingly, in various embodiments, a medical device such as a micropump or patch device can include a reservoir containing a pharmaceutical formulation, a subcutaneous injection needle configured for removable insertion into skin of a patient, a micropump having an inlet in fluid communication with the reservoir and an outlet in fluid communication with the subcutaneous injection needle, a control system configured for controlling the micropump to deliver the pharmaceutical formulation from the reservoir to the subcutaneous injection needle, whereby the pharmaceutical formulation is administered subcutaneously to a patient, and a housing for supporting the reservoir, subcutaneous injection needle, micropump and control system, the housing being portable and adapted for contact with the skin of the patient. The liquid pharmaceutical formulation contained within the reservoir can be any of the liquid pharmaceutical formulations or the unit liquid pharmaceutical formulations described herein.

In certain embodiments, the medical device can be of a unitary construction. Such medical devices can be for a single or one-time use. In particular embodiments, the medical device can be of a multi-piece construction. In such medical devices, a disposable or a reusable portion or component can be present. For example, a housing defining or including the reservoir can be a disposable or a reusable component of the medical device. In some embodiments, the disposable or reusable housing defining or including the reservoir can contain a pharmaceutical formulation of the present teachings. In various embodiments, the subcutaneous injection needle can be a disposable component of the medical device.

In certain embodiments, the medical device is selected from the group comprising a needle and syringe set, an autoinjector, a single use fixed dose injection pen, a multiuse fixed dose injection pen, a single use variable dose injection pen, a patient-controlled injector, a safety syringe, or a multiuse variable dose injection pen.

### Time of Administration

In certain embodiments, the liquid pharmaceutical formulations described herein are administered to the patient by subcutaneous injection over a period of equal to or less than 30 secs (e.g., 1 sec, 2 secs, 3 secs, 4 secs, 5 secs, 6 secs, 7 secs, 8 secs, 9 secs, 10 secs, 11 secs, 12 secs, 13 secs, 14 secs, 15 secs, 16 secs, 17 secs, 18 secs, 19 secs, 20 secs, 21 secs, 22 secs, 23 secs, 24 secs, 25 secs, 26 secs, 27 secs, 28 secs, 29 secs, or 30 secs).

### Patients

In certain embodiments, the human is an adult human. In certain embodiments, the adult human has New York Heart Association (NYHA) Class II, Class Ill or Class IV chronic heart failure, liver disease or impairment, or renal disease or impairment. In certain embodiments, the adult human displays reduced responsiveness to oral diuretics before beginning subcutaneous administration of a loop diuretic according to a method described herein.

In certain embodiments, the adult human is 30 to 80 years of age, 35 to 80 years of age, 40 to 80 years of age, 45 to 80 years of age, 50 to 80 years of age, 55 to 80 years of age, 60 to 80 years of age, 65 to 80 years of age, 70 to 80 years of age, 75 to 80 years of age, 30 to 75 years of age, 30 to 70 years of age, 30 to 65 years of age, 30 to 60 years of age, 30 to 55 years of age, 30 to 50 years of age, 30 to 45 years of age, 30 to 40 years of age, 30 to 35 years of age, 35 to 75 years of age, 35 to 70 years of age, 35 to 65 years of age, 35 to 60 years of age, 35 to 55 years of age, 35 to 50 years of age, 35 to 45 years of age, 35 to 40 years of age, 40 to 75 years of age, 40 to 70 years of age, 40 to 65 years of age, 40 to 60 years of age, 40 to 55 years of age, 40 to 50 years of age, 40 to 45 years of age, 45 to 75 years of age, 45 to 70 years of age, 45 to 65 years of age, 45 to 60 years of age, 45 to 55 years of age, 45 to 50 years of age, 50 to 75 years of age, 50 to 70 years of age, 50 to 65 years of age, 50 to 60 years of age, 50 to 55 years of age, 55 to 75 years of age, 55 to 70 years of age, 55 to 65 years of age, 55 to 60 years of age, 60 to 75 years of age, 60 to 70 years of age, 60 to 65 years of age, 65 to 75 years of age, 65 to 70 years of age, or 70 to 75 years of age. In certain embodiments, the adult human is 30 to 80 years of age. In certain embodiments, the adult human is 45 to 80 years of age.

In certain embodiments, the adult human is 30, 31, 32, 33, 34, 35, 36, 37, 38, 39, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 years of age.

When administered for the treatment or prevention of a disease or disorder disclosed herein, it may be understood that an effective dosage can vary depending upon many factors such as the particular compound or therapeutic combination utilized, the mode of administration, and severity of the condition being treated, as well as the various physical factors related to the individual being treated. In therapeutic applications, a liquid pharmaceutical formulation described herein may be provided to a patient already suffering from said disease or disorder in an amount sufficient to cure or at least partially ameliorate the symptoms of the disease or disorder and its complications. The dosage to be used in the treatment of a specific individual typically must be subjectively determined by an attending physician. The variables involved include the specific condition and state as well as the size, age and response pattern of the patient. In some embodiments, the amount sufficient to cure or at least partially ameliorate the symptoms of the disease or disorder and its complications is an effective amount. In some embodiments, the amount sufficient to cure or at least partially ameliorate the symptoms of the disease or disorder and its complications is an effective amount.

### EXAMPLES

The methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1. Furosemide Solubility Studies

A Design of Experiment (DOE) study was executed to evaluate eight different pharmaceutical buffers and surfactants with and without benzyl alcohol in order to identify any promising combinations to improve furosemide solubility. The buffers and solubilizers listed in Table 1 were examined.

TABLE 1

| Buffers | Solubilizers |
|---|---|
| Tromethane (Tris) | Polysorbate 20 (PS20) |
| Sodium phosphate | Polysorbate 80 (PS80) |
| Ammonium acetate | Polyethylene glycol (PEG) |
| Sodium carbonate | Polyvinylpyrrolidone (PVP) |
| Sodium citrate | N-Methyl-2-pyrrodlidine (NMP) |
| Potassium phosphate | Ethanol |
| Arginine HCl | Propylene glycol |
| Glycine HCl | Glycerin |
| Sodium tetraborate | Hydroxypropyl-β-cyclodextrin |
| Potassium tetraborate | |

The vehicle formulations listed in Table 2 were made with all buffers at 50 mM and all solubilizers except benzyl alcohol at 1 wt %. Formulations were made with and without benzyl alcohol at 3.5 wt %.

TABLE 2

| | | Solubilizer | Benzyl | pH |
|---|---|---|---|---|
| # | Buffer at 50 mM | at 1% | Alcohol % | Adjuster |
| 1 | Tris | None | 3.5 | NaOH |
| 2 | Tris | PEG 400 | 3.5 | NaOH |
| 3 | Tris | Ethanol | 3.5 | NaOH |
| 4 | Tris | NMP | 0 | NaOH |
| 5 | Sodium Citrate | Glycerin | 0 | NaOH |
| 6 | Sodium Citrate | Propylene Glycol | 3.5 | NaOH |
| 7 | Sodium Citrate | PS20 | 0 | NaOH |
| 8 | Potassium Phosphate | Glycerin | 0 | NaOH |
| 9 | Potassium Phosphate | PS80 | 3.5 | KOH |
| 10 | Potassium Phosphate | PVP K15 | 0 | KOH |
| 11 | Sodium Phosphate | PS20 | 0 | NaOH |
| 12 | Sodium Bicarbonate | PS20 | 3.5 | NaOH |
| 13 | Potassium Phosphate | Glycerin | 0 | KOH |
| 14 | Tris | PS20 | 3.5 | NaOH |
| 15 | Potassium Phosphate | PS80 | 3.5 | NaOH |
| 16 | Potassium Phosphate | PVP K15 | 0 | NaOH |
| 17 | Sodium Phosphate | PVP K15 | 3.5 | NaOH |
| 18 | Sodium Phosphate | Ethanol | 0 | NaOH |
| 19 | Sodium Bicarbonate | PS80 | 0 | NaOH |
| 20 | Sodium Bicarbonate | NMP | 3.5 | NaOH |
| 21 | Arginine HCl | Propylene Glycol | 3.5 | NaOH |
| 22 | Arginine HCl | PEG 400 | 0 | NaOH |
| 23 | Arginine HCl | PVP K15 | 0 | NaOH |
| 24 | Glycine HCl | PEG 400 | 3.5 | NaOH |
| 25 | Glycine HCl | Ethanol | 0 | NaOH |
| 26 | Glycine HCl | NMP | 3.5 | NaOH |
| 27 | Ammonium Acetate | Glycerin | 3.5 | NaOH |
| 28 | Ammonium Acetate | PS80 | 3.5 | NaOH |
| 29 | Ammonium Acetate | Propylene Glycol | 0 | NaOH |
| 30 | Tris | None | 0 | NaOH |
| 31 | Sodium Phosphate | None | 0 | NaOH |
| 32 | Ammonium Acetate | None | 0 | NaOH |
| 33 | Sodium Bicarbonate | None | 0 | NaOH |
| 34 | Sodium Citrate | None | 0 | NaOH |
| 35 | Potassium Phosphate | None | 0 | NaOH |
| 36 | Arginine | None | 0 | NaOH |
| 37 | Glycine | None | 0 | NaOH |

Vehicle formulations were prepared with the excipients as listed in Table 2. Furosemide (0.75 g) was added to an amber glass vial along with 5 mL of vehicle solution. It was mixed for 30 min and then the pH was adjusted to pH 7-8 with 5M NaOH (or 5M KOH for selected formulations) and 37% HCl. The solid form visually showed a transition from white slurry suspension to a new morphology. It was confirmed whether excess solid was present in the mixture, and if not, furosemide was added in increments till there was excess. Samples were warmed to 25.0° C. and mixed by rocking and rotating to facilitate dissolution. After 7 days, the sample was removed and equilibrated to room temperature. Then 1.5 mL of sample was removed and filtered using a PVDF 0.2 μm syringe filter. The first two drops of the filtrate were discarded. The filtrate was tested for pH, osmolality, and furosemide concentration. The results are shown in Table 3.

TABLE 3

| Sample | | Benzyl | Secondary | pH | 7 d | 7 d Conc | 7 d |
|---|---|---|---|---|---|---|---|
| Set | Buffer | alcohol | Solubilizer | Adjuster | pH | (mg/mL) | Osmo |
| 1 | Tris | 3.5 | None | NaOH | 7.77 | 94.48 | 319 |
| 1 | Tris | 3.5 | None | NaOH | 7.57 | 94.24 | 320 |
| 1 | Tris | 3.5 | None | NaOH | 7.65 | 95.57 | 320 |
| 1 | Tris | 3.5 | PEG400 | NaOH | 7.89 | 97.57 | 346 |
| 1 | Tris | 3.5 | PEG400 | NaOH | 7.72 | 98.43 | 343 |
| 1 | Tris | 3.5 | PEG400 | NaOH | 7.78 | 98.57 | 344 |
| 1 | Tris | 3.5 | Ethanol | NaOH | 7.80 | 94.13 | 319 |
| 1 | Tris | 3.5 | Ethaol | NaOH | 7.60 | 96.13 | 324 |
| 1 | Tris | 3.5 | Ethanol | NaOH | 7.55 | 96.68 | 325 |
| 1 | Tris | 0 | NMP | NaOH | 7.34 | 65.78 | 320 |
| 1 | Tris | 0 | NMP | NaOH | 7.21 | 66.53 | 318 |
| 1 | Tris | 0 | NMP | NaOH | 7.58 | 65.20 | 320 |
| 1 | Sodium | 0 | Glycerin | NaOH | 7.40 | 26.75 | 362 |
| | Citrate | | | | | | |
| 1 | Sodium | 0 | Glycerin | NaOH | 7.64 | 26.95 | 351 |
| | Citrate | | | | | | |
| 1 | Sodium | 0 | Glycerin | NaOH | 7.47 | 27.36 | 359 |
| | Citrate | | | | | | |
| 2 | Sodium | 3.5 | Propylene | NaOH | 7.49 | 60.27 | 482 |
| | Citrate | | Glycol | | | | |
| 2 | Sodium | 3.5 | Propylene | NaOH | 7.41 | 61.72 | 475 |
| | Citrate | | Glycol | | | | |
| 2 | Sodium | 3.5 | Propylene | NaOH | 7.33 | 61.62 | 470 |
| | Citrate | | Glycol | | | | |
| 2 | Sodium | 0 | PS20 | NaOH | 7.11 | 30.54 | 260 |
| | Citrate | | | | | | |
| 2 | Sodium | 0 | PS20 | NaOH | 7.08 | 30.49 | 261 |
| | Citrate | | | | | | |
| 2 | Sodium | 0 | PS20 | NaOH | 7.09 | 30.66 | 257 |
| | Citrate | | | | | | |
| 2 | Potassium | 0 | Glycerin | NaOH | 7.51 | 40.84 | 379 |
| | Phosphate | | | | | | |
| 2 | Potassium | 0 | Glycerin | NaOH | 7.49 | 40.20 | 376 |
| | Phosphate | | | | | | |
| 2 | Potassium | 0 | Glycerin | NaOH | 7.46 | 40.49 | 376 |
| | Phosphate | | | | | | |
| 2 | Potassium | 3.5 | PS80 | KOH | 7.36 | 133.91 | 458 |
| | Phosphate | | | | | | |
| 2 | Potassium | 3.5 | PS80 | KOH | 7.39 | 131.09 | 450 |
| | Phosphate | | | | | | |
| 2 | Potassium | 3.5 | PS80 | KOH | 7.39 | 134.74 | 463 |
| | Phosphate | | | | | | |
| 2 | Potassium | 0 | PVP K15 | KOH | 7.81 | 61.85 | 331 |
| | Phosphate | | | | | | |
| 2 | Potassium | 0 | PVP K15 | KOH | 7.45 | 64.80 | 335 |
| | Phosphate | | | | | | |
| 2 | Potassium | 0 | PVP K15 | KOH | 7.33 | 65.37 | 335 |
| | Phosphate | | | | | | |
| 2 | Sodium | 0 | PS20 | NaOH | 7.21 | 36.00 | 251 |
| | Phosphate | | | | | | |
| 2 | Sodium | 0 | PS20 | NaOH | 7.35 | 35.33 | 249 |
| | Phosphate | | | | | | |
| 2 | Sodium | 0 | PS20 | NaOH | 7.23 | 36.15 | 248 |
| | Phosphate | | | | | | |
| 2 | Sodium | 3.5 | PS20 | NaOH | 8.27 | 72.13 | 328 |
| | Bicarbonate | | | | | | |
| 2 | Sodium | 3.5 | PS20 | NaOH | 8.03 | 76.40 | 300 |
| | Bicarbonate | | | | | | |
| 2 | Sodium | 3.5 | PS20 | NaOH | 8.24 | 74.28 | 304 |
| | Bicarbonate | | | | | | |
| 2 | Potassium | 0 | Glycerin | KOH | 7.51 | 57.11 | 421 |
| | Phosphate | | | | | | |
| 2 | Potassium | 0 | Glycerin | KOH | 7.44 | 58.65 | 422 |
| | Phosphate | | | | | | |
| 2 | Potassium | 0 | Glycerin | KOH | 7.53 | 57.48 | 422 |
| | Phosphate | | | | | | |
| 3 | Tris | 3.5 | PS20 | NaOH | 7.25 | 103.43 | 317 |
| 3 | Tris | 3.5 | PS20 | NaOH | 7.00 | 104.80 | 313 |
| 3 | Potassium | 3.5 | PS80 | NaOH | 7.48 | 99.36 | 410 |
| | Phosphate | | | | | | |
| 3 | Potassium | 3.5 | PS80 | NaOH | 7.29 | 100.08 | 407 |
| | Phosphate | | | | | | |
| 3 | Potassium | 0 | PVP K15 | NaOH | 7.35 | 51.07 | 311 |
| | Phosphate | | | | | | |
| 3 | Potassium | 0 | PVP K15 | NaOH | 7.33 | 50.63 | 308 |
| | Phosphate | | | | | | |
| 3 | Sodium | 3.5 | PVP K15 | NaOH | 7.38 | 74.82 | 362 |
| | Phosphate | | | | | | |
| 3 | Sodium | 3.5 | PVP K15 | NaOH | 7.84 | 75.61 | 363 |
| | Phosphate | | | | | | |
| 3 | Sodium | 0 | Ethanol | NaOH | 7.10 | 32.69 | 249 |
| | Phosphate | | | | | | |
| 3 | Sodium | 0 | Ethanol | NaOH | 7.11 | 32.98 | 247 |
| | Phosphate | | | | | | |
| 3 | Sodium | 0 | PS80 | NaOH | 7.51 | 46.87 | 212 |
| | Bicarbonate | | | | | | |
| 3 | Sodium | 0 | PS80 | NaOH | 7.42 | 46.90 | 212 |
| | Bicarbonate | | | | | | |
| 3 | Sodium | 3.5 | NMP | NaOH | 7.98 | 87.89 | 388 |
| | Bicarbonate | | | | | | |
| 3 | Sodium | 3.5 | NMP | NaOH | 7.97 | 87.86 | 391 |
| | Bicarbonate | | | | | | |
| 3 | Arginine | 3.5 | Propylene | NaOH | 7.34 | 107.83 | 438 |
| | | | Glycol | | | | |
| 3 | Arginine | 3.5 | Propylene | NaOH | 7.02 | 110.38 | 428 |
| | | | Glycol | | | | |
| 3 | Arginine | 0 | PEG 400 | NaOH | 6.96 | 70.93 | 275 |
| 3 | Arginine | 0 | PEG 400 | NaOH | 7.35 | 70.98 | 269 |
| 3 | Arginine | 0 | PVP K15 | NaOH | 7.11 | 76.46 | 251 |
| 3 | Arginine | 0 | PVP K15 | NaOH | 7.04 | 75.73 | 260 |
| 4 | Glycine | 3.5 | PEG400 | NaOH | 6.77 | 88.65 | 358 |
| 4 | Glycine | 3.5 | PEG400 | NaOH | 7.43 | 89.12 | 358 |
| 4 | Glycine | 0 | Ethanol | NaOH | 7.07 | 45.14 | 249 |
| 4 | Glycine | 0 | Ethanol | NaOH | 7.05 | 47.33 | 248 |
| 4 | Glycine | 3.5 | NMP | NaOH | 7.27 | 93.16 | 412 |
| 4 | Glycine | 3.5 | NMP | NaOH | 7.54 | 94.90 | 409 |
| 4 | Ammonium | 3.5 | Glycerin | NaOH | 7.22 | 88.54 | 476 |
| | Acetate | | | | | | |
| 4 | Ammonium | 3.5 | Glycerin | NaOH | 7.27 | 89.47 | 471 |
| | Acetate | | | | | | |
| 4 | Ammonium | 3.5 | PS80 | NaOH | 7.66 | 86.42 | 378 |
| | Acetate | | | | | | |
| 4 | Ammonium | 3.5 | PS80 | NaOH | 7.45 | 87.61 | 377 |
| | Acetate | | | | | | |
| 4 | Ammonium | 0 | Propylene | NaOH | 7.59 | 48.21 | 403 |
| | Acetate | | Glycol | | | | |
| 4 | Ammonium | 0 | Propylene | NaOH | 7.11 | 48.78 | 404 |
| | Acetate | | Glycol | | | | |
| Buffer | Tris | 0 | None | NaOH | 7.21 | 43.32 | 327 |
| only | | | | | | | |
| Buffer | Tris | 0 | None | NaOH | 7.18 | 52.70 | 286 |
| only | | | | | | | |
| Buffer | Sodium | 0 | None | NaOH | 7.30 | 32.57 | 251 |
| only | Phosphate | | | | | | |
| Buffer | Sodium | 0 | None | NaOH | 7.41 | 33.90 | 243 |
| only | Phosphate | | | | | | |
| Buffer | Ammonium | 0 | None | NaOH | 7.45 | 47.46 | 291 |
| only | Acetate | | | | | | |
| Buffer | Ammonium | 0 | None | NaOH | 7.48 | 51.39 | 281 |
| only | Acetate | | | | | | |
| Buffer | Sodium | 0 | None | NaOH | 7.99 | 41.36 | 229 |
| only | Bicarbonate | | | | | | |
| Buffer | Sodium | 0 | None | NaOH | 7.89 | 44.50 | 219 |
| only | Bicarbonate | | | | | | |
| Buffer | Sodium | 0 | None | NaOH | 7.71 | 25.56 | 273 |
| only | Citrate | | | | | | |
| Buffer | Sodium | 0 | None | NaOH | 7.43 | 27.66 | 261 |
| only | Citrate | | | | | | |
| Buffer | Potassium | 0 | None | NaOH | 7.29 | 33.75 | 259 |
| only | Phosphate | | | | | | |
| Buffer | Potassium | 0 | None | NaOH | 7.32 | 33.12 | 255 |
| only | Phosphate | | | | | | |
| Buffer | Arginine | 0 | None | NaOH | 7.42 | 56.95 | 295 |
| only | | | | | | | |
| Buffer | Arginine | 0 | None | NaOH | 7.14 | 57.40 | 293 |
| only | | | | | | | |
| Buffer | Glycine | 0 | None | NaOH | 7.57 | 46.48 | 248 |
| only | | | | | | | |
| Buffer | Glycine | 0 | None | NaOH | 7.67 | 46.38 | 247 |
| only | | | | | | | |

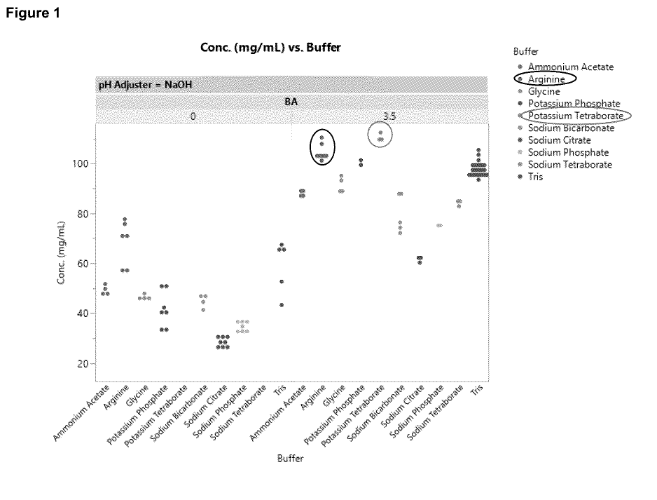

Results were analyzed statistically using JMP statistical analysis software (Version 18.0.1) to find which excipients positively impacted the solubility of furosemide in solution. When the 7-day furosemide concentration of all formulations adjusted with NaOH is plotted, formulations with arginine buffer have higher concentrations of furosemide compared to similar formulations with and without benzyl alcohol (FIG. 1).

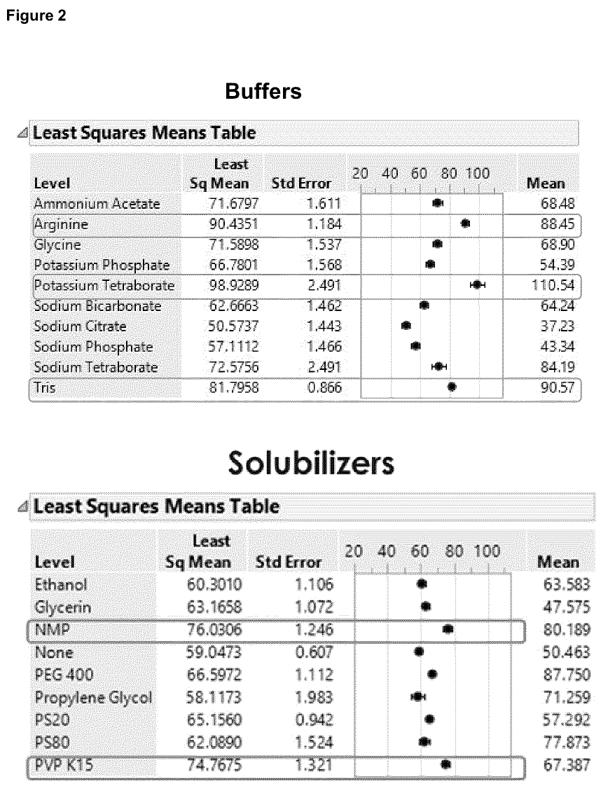

If a fit model least squares is run with buffer, benzyl alcohol, and secondary solubilizer as the variables, the impact of each variable can be seen within the DOE from the least squares means tables shown in FIG. 2 for buffers and secondary solubilizers (not including benzyl alcohol). Surprisingly, the arginine buffer appears to have a significant impact on solubility that may be greater than Tris buffer.

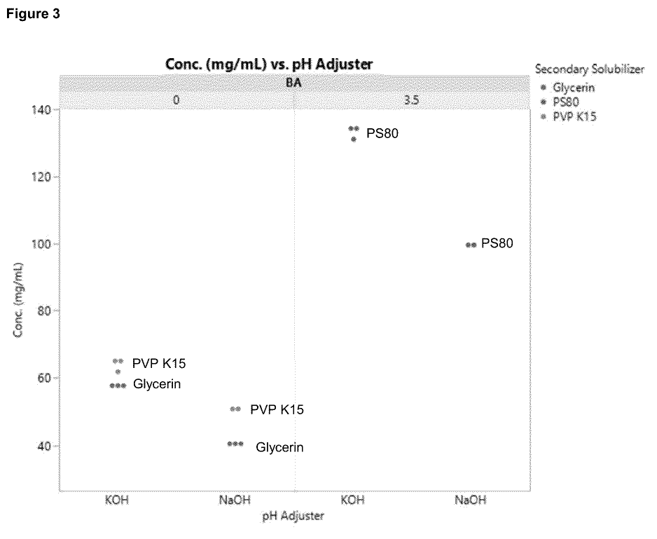

If the potassium phosphate buffer formulations adjusted with either KOH of NaOH are plotted, KOH leads to significantly improved solubility compared to NaOH with or without benzyl alcohol (FIG. 3).

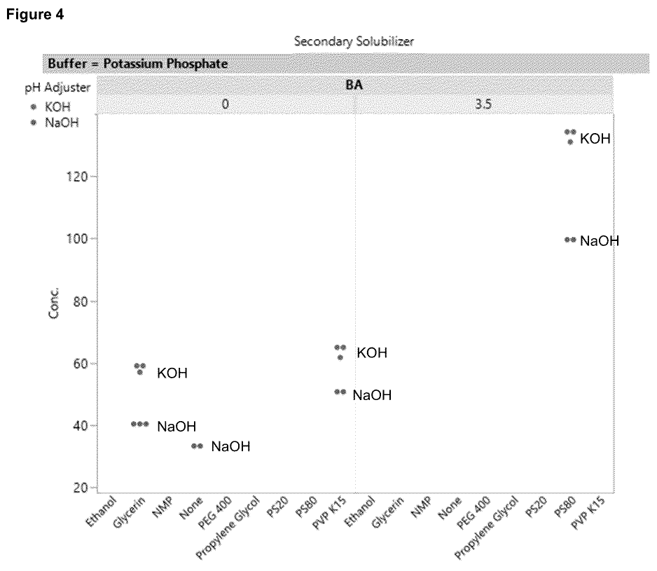

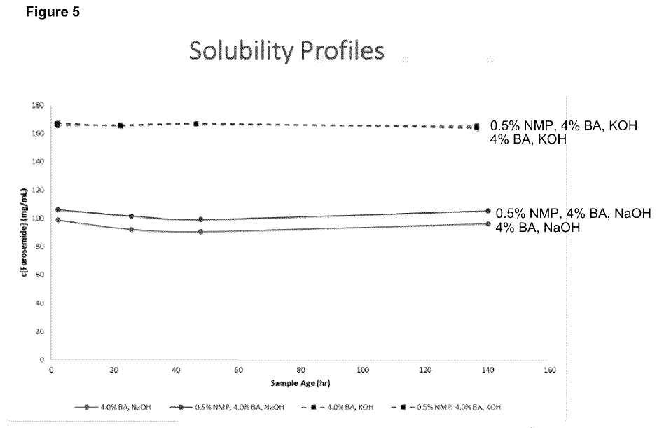

The effect of pH adjusters in formulations with secondary solubilizers with or without benzyl alcohol was also examined in FIG. 4. The use of pH adjusters in formulations with benzyl alcohol greatly increased the concentration of furosemide that dissolved in the formulations. These formulations were stable over the monitored time period (up to 140 hours) as shown in FIG. 5.

Additional furosemide solubility studies were conducted with the formulations in Table 4 where Na₂B₄O₇ refers to sodium tetraborate and K₂B₄O₇ refers to potassium tetraborate. Example 4 comprises arginine and hydroxypropyl-β-cyclodextrin.

TABLE 4

| | | | | | | Replicates of |
|---|---|---|---|---|---|---|
| Ex. No. | BnOH | Na₂B₄O₇ | K₂B₄O₇ | Arginine | Tris | Solubility Samples |
| Control | 3.5% | N/A | N/A | N/A | 50 mM | 1 |
| 1 | 3.5% | 50 mM | N/A | N/A | N/A | 3 |
| 2 | 3.5% | N/A | 50 mM | N/A | N/A | 3 |
| 3 | 3.5% | N/A | N/A | 50 mM | N/A | 3 |
| 4 | 3.5% | N/A | N/A | 50 mM | 150 mg/mL HP-β- | 3 |
| | | | | | cyclodextrin | |

Solubility study samples were prepared as follows:
(1) Vehicle formulations were prepared with the excipients as listed in Table 4. Furosemide (1.50 g) was added to a 10 mL amber glass vial along with 10 mL of vehicle solution. For each solubility study group, a control sample was prepared, and three replicates of each formulation condition were prepared.
(2) The solutions were mixed for 30 min and then adjusted to pH 7-8. The presence of excess solid in the mixture was confirmed. If excess solid was not present, more furosemide was added in increments until there was excess. The pH was adjusted again to have the solid form transition and ensure there was excess solid present, if a second addition of furosemide was performed.
(3) Samples were warmed to 25.0° C. and mixed by rocking and rotating to facilitate dissolution. After 7 days, the sample was removed and equilibrated to room temperature. Then 1.5 mL of sample was removed and filtered using a PVDF 0.2 μm syringe filter. The first two drops of the filtrate were discarded. The filtrate was tested for pH, osmolality, and furosemide concentration. Samples were placed at 25.0° C. and mixed by rocking and rotating to facilitate dissolution. After 7 days, the samples were removed and equilibrated to room temperature.
(4) 2 mL of the sample was removed and filtered using a PVDF 0.2 μm syringe filter. The first two drops of the filtrate were discarded.
(5) The filtrate was tested for pH, osmolality, furosemide soluble concentration, and impurities %.

The furosemide soluble concentration, pH, and osmolality of the upper clear solution filtrates in the solubility study samples at equilibrium at the t7d time point is summarized in Table 5. Benzyl alcohol concentration and benzaldehyde concentration are summarized in Table 6. At t7d, the furosemide soluble concentration in all tested buffer system-additional solubilizer combinations were >80 mg/mL.

TABLE 5

| | Buffer | | pH | 7 d | Furosemide | Osmolality |
|---|---|---|---|---|---|---|
| Ex. No. | (50 mM) | BA | Adjuster | pH | Conc. (mg/mL) | (mOsm/kg) |
| Control | Tris | 3.5% | NaOH | 7.32 | 94.60 | 315 |
| 1 | Na₂B₄O₇ | 3.5% | NaOH | 7.51 | 82.82 | 475 |
| 1 | Na₂B₄O₇ | 3.5% | NaOH | 7.25 | 84.38 | 477 |
| 1 | Na₂B₄O₇ | 3.5% | NaOH | 7.27 | 85.37 | 475 |
| 2 | K₂B₄O₇ | 3.5% | NaOH | 7.49 | 109.60 | 514 |
| 2 | K₂B₄O₇ | 3.5% | NaOH | 7.11 | 109.64 | 517 |
| 2 | K₂B₄O₇ | 3.5% | NaOH | 6.89 | 112.39 | 518 |
| 3 | Arginine | 3.5% | NaOH | 7.28 | 102.13 | 310 |
| 3 | Arginine | 3.5% | NaOH | 7.26 | 102.15 | 311 |
| 3 | Arginine | 3.5% | NaOH | 7.89 | 101.07 | 318 |
| 4* | Arginine | 3.5% | NaOH | 7.75 | 102.22 | 458 |
| 4* | Arginine | 3.5% | NaOH | 8.03 | 102.37 | 458 |
| 4* | Arginine | 3.5% | NaOH | 7.99 | 101.68 | 459 |

*Samples 4 have 150 mg/mL hydroxypropyl-β-cyclodextrin in addition to the excipients listed.

TABLE 6

| | | | | Benzyl | Benzal- |
|---|---|---|---|---|---|
| | Buffer | | pH | Alcohol | dehyde |
| Ex. No. | (50 mM) | BA | Adjuster | (mg/mL) | (mg/mL) |
| Control | Tris | 3.5% | NaOH | 31.32 | ND |
| 1 | Na₂B₄O₇ | 3.5% | NaOH | 31.81 | ND |
| 1 | Na₂B₄O₇ | 3.5% | NaOH | 32.26 | ND |
| 1 | Na₂B₄O₇ | 3.5% | NaOH | 32.63 | ND |
| 2 | K₂B₄O₇ | 3.5% | NaOH | 31.68 | ND |
| 2 | K₂B₄O₇ | 3.5% | NaOH | 30.87 | ND |
| 2 | K₂B₄O₇ | 3.5% | NaOH | 30.97 | ND |
| 3 | Arginine | 3.5% | NaOH | 31.28 | ND |
| 3 | Arginine | 3.5% | NaOH | 31.64 | ND |
| 3 | Arginine | 3.5% | NaOH | 31.51 | ND |
| 4* | Arginine | 3.5% | NaOH | 32.02 | ND |
| 4* | Arginine | 3.5% | NaOH | 32.16 | ND |
| 4* | Arginine | 3.5% | NaOH | 32.39 | ND |

ND = Not detected
*Samples 4 have 150 mg/mL hydroxypropyl-β-cyclodextrin in addition to the excipients listed.
pH: Most samples that were adjusted to be between pH 7-8 during the preparation of the solubility samples stayed within this range at the t 7 d time point.

Furosemide Soluble Concentration: The furosemide soluble concentration in all tested buffer system-secondary solubilizer combinations were >80 mg/mL. The following formulations showed substantially increased furosemide soluble concentration compared to the control formulation:
50 mM potassium tetraborate, 3.5% benzyl alcohol, pH 7-8 (t7d average at 110.5 mg/mL, 16.8% increase from Control)
50 mM arginine, 3.5% benzyl alcohol, pH 7-8 (t7d average at 101.8 mg/mL, 7.6% increase from Control)
50 mM arginine, 3.5% benzyl alcohol, 150 mg/mL hydroxypropyl-β-cyclodextrin, pH 7-8 (t7d average at 102.1 mg/mL, 9.2% increase from Control)

Impurities: Impurity percentage results were low in all solubility study t7d samples. A common furosemide impurity, furfural, percentage was below limit of detection. Related Compound A and Related Compound B are also known furosemide impurities. Related Compound A was not detected. Related Compound B was the only impurity to be detected above the 0.05% reportable limit.

### Example 2:60° C. Accelerated Condition Stability Study

A 12-week stability study at accelerated 60° C. condition was performed. For formulations with a day 7 (t7d) furosemide soluble concentration larger than 80 mg/mL, enough remaining solution was filtered from the sample to mostly fill a 2 mL amber glass vial. For each solubility study t7d sample, one vial of sample was prepared for each time point, making an N=3 sample size for each formulation at each time point for the accelerated condition stability study (except for the control, which was N=1). Vials were placed in an oven at 60° C. while being protected from light. After 4, 8, 12 weeks, samples were tested for furosemide soluble concentration, pH, appearance, and impurities.

The furosemide assay results are summarized in Table 7. The pH testing results are summarized in Table 8.

TABLE 7

| | | | Furosemide (mg/mL), 60° C. | | |
|---|---|---|---|---|---|
| Ex. No. | Formulation | t0 | 4 wk | 8 wk | 12 wk |
| Control | 50 mM Tris, 3.5% BA | 94.6 | 95.05 | 91.79 | 90.03 |
| 1 | 50 mM Na₂B₄O₇, 3.5% BA | 82.82 | 80.23 | 80.35 | 79.3 |
| 1 | 50 mM Na₂B₄O₇, 3.5% BA | 84.38 | 84.04 | 81.5 | 79.54 |
| 1 | 50 mM Na₂B₄O₇, 3.5% BA | 85.37 | 83.98 | 81.79 | 79.38 |
| 2 | 50 mM K₂B₄O₇, 3.5% BA | 109.6 | 106.58 | 101.87 | 101.87 |
| 2 | 50 mM K₂B₄O₇, 3.5% BA | 109.64 | 110.15 | 106.96 | 104.33 |
| 2 | 50 mM K₂B₄O₇, 3.5% BA | 112.39 | 107.53 | 105.96 | 104.33 |
| 3 | 50 mM arginine, 3.5% BA | 102.13 | 99.51 | 97.67 | 97.61 |
| 3 | 50 mM arginine, 3.5% BA | 102.58 | 101.07 | 99.24 | 97.61 |
| 3 | 50 mM arginine, 3.5% BA | 101.07 | 102.85 | 98.53 | 98.41 |
| 4 | 50 mM arginine, 3.5% BA, 150 | 102.22 | 98.51 | 100.07 | 97.76 |
| | mg/mL HP-β-cyclodextrin | | | | |
| 4 | 50 mM arginine, 3.5% BA, 150 | 102.37 | 98.45 | 100.83 | 97.19 |
| | mg/mL HP-β-cyclodextrin | | | | |
| 4 | 50 mM arginine, 3.5% BA, 150 | 101.68 | 98.49 | 99.69 | 97.38 |
| | mg/mL HP-β-cyclodextrin | | | | |

TABLE 8

| | | pH, 60° C. | | | |
|---|---|---|---|---|---|
| Ex. No | Vehicle Formulation | t0 | 4 wk | 8 wk | 12 wk |
| Control | 50 mM Tris, 3.5% BA | 7.32 | 7.02 | 6.96 | 6.77 |
| 1 | 50 mM Na₂B₄O₇, 3.5% BA | 7.51 | 7.37 | 7.36 | 7.19 |
| 1 | 50 mM Na₂B₄O₇, 3.5% BA | 7.25 | 7.06 | 7.11 | 6.88 |
| 1 | 50 mM Na₂B₄O₇, 3.5% BA | 7.27 | 7.13 | 7.16 | 6.97 |
| 2 | 50 mM K₂B₄O₇, 3.5% BA | 7.49 | 7.24 | 7.31 | 7.11 |
| 2 | 50 mM K₂B₄O₇, 3.5% BA | 7.11 | 6.87 | 6.81 | 6.72 |
| 2 | 50 mM K₂B₄O₇, 3.5% BA | 6.89 | 6.7 | 6.65 | 6.54 |
| 3 | 50 mM arginine, 3.5% BA | 7.28 | 6.91 | 6.84 | 6.73 |
| 3 | 50 mM arginine, 3.5% BA | 7.26 | 6.91 | 6.84 | 6.71 |
| 3 | 50 mM arginine, 3.5% BA | 7.89 | 7.33 | 7.25 | 7.03 |
| 4 | 50 mM arginine, 3.5% BA, 150 | 7.75 | 6.95 | 6.88 | 6.83 |
| | mg/mL HP-β-cyclodextrin | | | | |
| 4 | 50 mM arginine, 3.5% BA, 150 | 8.03 | 7.12 | 7.17 | 7.06 |
| | mg/mL HP-β-cyclodextrin | | | | |
| 4 | 50 mM arginine, 3.5% BA, 150 | 7.99 | 6.93 | 7.07 | 6.89 |
| | mg/mL HP-β-cyclodextrin | | | | |

Appearance: The appearance of the accelerated 60° C. stability study samples were evaluated at each time point. Stability samples, which appeared pale yellow at t0, gradually turned to light orange to light brown at t4 wk and t8 wk, and brown at t12 wk at 60° C. All samples remained clear over the 12 weeks with no visible particulate matter found in any sample at any time point. At each time point, especially early time points, variation in coloration among samples was observed. A potential correlation between the darkness of the coloration and impurity % level was observed.

Furosemide Assay: The furosemide concentration in the stability study samples decreased over time. From t0 to t12 wk at 60° C., a 3-7% decrease was observed in all formulations. The following formulations remain to have the highest furosemide concentration at t12 wk:
50 mM potassium tetraborate, 3.5% benzyl alcohol, pH 7-8 (t12 wk 103.3 mg/mL)
50 mM arginine, 3.5% benzyl alcohol, pH 7-8 (t12 wk 97.8 mg/mL)
50 mM arginine, 3.5% benzyl alcohol, 150 mg/mL hydroxypropyl-β-cyclodextrin, pH 7-8 (t12 wk 97.4 mg/mL)

Impurities: The known impurity Related Compound B was found to grow in impurity % over time in the 12-week 60° C. stability study. Moreover, a correlation between the formulation pH and the Related Compound B impurity % was found. Furfural impurity % grew from t0 to t4 wk, but eventually showed a trend of slight decrease from t4 wk to t12 wk. Unknown impurities such as RRT 0.21, RRT 0.94, RRT 1.13, RRT 1.21 showed growth in impurity % from t0 to t12 wk. Formulations with arginine as the buffer had two noticeably growing unknown impurities that were specific to the formulation, RRT 0.81 and RRT 0.82, likely related to arginine degradation. Total impurities % increased overtime from t0 to t12 wk in all stability samples (from Not detected-0.10% at t0 to 1.44-3.43% at t12 wk), with no outstanding stabilizing/destabilizing effect found in any formulations compared to the control formulation.

Tris is the widely accepted buffer for formulations of furosemide. It was therefore surprising to discover that unconventional buffers for furosemide formulations, such as arginine and potassium tetraborate, performed similarly, if not better, than Tris in solubility and stability studies where so many other buffers failed (see, e.g., FIG. 1, which demonstrates that arginine and potassium tetraborate exhibited furosemide solubility comparable to Tris, whereas all other buffers performed worse than Tris).

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. A liquid pharmaceutical formulation comprising:
(i) furosemide, or a pharmaceutically acceptable salt thereof;
(ii) a solubilizer; and
(iii) a pharmaceutically acceptable buffer selected from the group consisting of ammonium acetate, sodium bicarbonate, arginine, sodium tetraborate, potassium tetraborate, and glycine.

2. The liquid pharmaceutical formulation of claim 1, wherein the pharmaceutically acceptable buffer is an arginine buffer or a potassium tetraborate buffer.

3. The liquid pharmaceutical formulation of claim 1, wherein the pharmaceutically acceptable buffer is arginine HCl.

4. (canceled)

5. The liquid pharmaceutical formulation of claim 1, wherein the concentration of pharmaceutically acceptable buffer in the liquid pharmaceutical formulation is from about 10 mM to 100 mM.

6. The liquid pharmaceutical formulation of claim 1, wherein the concentration of pharmaceutically acceptable buffer in the liquid pharmaceutical formulation is from about 25 mM to 75 mM.

7. (canceled)

8. The liquid pharmaceutical formulation of claim 1, wherein the solubilizer is selected from the group consisting of a polysorbate, benzyl alcohol, polyethylene glycol, polyvinylpyrrolidone, N-methyl-2-pyrrolidone, ethanol, propylene glycol, and glycerin, or a combination thereof.

9. The liquid pharmaceutical formulation of claim 1, wherein the solubilizer is benzyl alcohol, polyvinylpyrrolidone, or N-methyl-2-pyrrolidone.

10-11. (canceled)

12. The liquid pharmaceutical formulation of claim 1, wherein the amount of solubilizer in the liquid pharmaceutical formulation is from 0.5% (w/w) to 5% (w/w).

13. The liquid pharmaceutical formulation of claim 1, wherein the amount of solubilizer in the liquid pharmaceutical formulation is about 1% (w/w).

14.-15. (canceled)

16. The liquid pharmaceutical formulation of claim 1, optionally further comprising:
(iv) a pH adjuster.

17. The liquid pharmaceutical formulation of claim 16, wherein the pH adjuster is potassium hydroxide or sodium hydroxide.

18. The liquid pharmaceutical formulation of claim 1, wherein the concentration of furosemide in the liquid pharmaceutical formulation is from about 1 mg/mL to about 250 mg/mL.

19. The liquid pharmaceutical formulation of claim 1, wherein the concentration of furosemide in the liquid pharmaceutical formulation is from about 40 mg/mL to about 200 mg/mL.

20. (canceled)

21. The liquid pharmaceutical formulation of claim 1, wherein the pH of the liquid pharmaceutical formulation is from about 6.5 to about 8.5.

22. (canceled)

23. A unit liquid pharmaceutical formulation comprising:
(i) from about 1 mg to about 250 mg of furosemide, or a pharmaceutically acceptable salt thereof;
(ii) from about 0.1% (w/w) to about 30% (w/w) of a solubilizer selected from the group consisting of a polysorbate, benzyl alcohol, polyethylene glycol, polyvinylpyrrolidone, N-methyl-2-pyrrolidone, ethanol, propylene glycol, and glycerin, or a combination thereof;
(iii) from about 10 mM to 100 mM of a pharmaceutically acceptable buffer selected from the group consisting of ammonium acetate, sodium bicarbonate, arginine, sodium tetraborate, potassium tetraborate, and glycine; and optionally
(iv) a pH adjuster that is potassium hydroxide or sodium hydroxide.

24. The liquid pharmaceutical formulation of claim 23 comprising:
(i) from about 1 mg to about 250 mg of furosemide, or a pharmaceutically acceptable salt thereof;
(ii) from about 0.1% (w/w) to about 30% (w/w) of N-methyl-2-pyrrolidone, benzyl alcohol, polyvinylpyrrolidone, or a combination thereof;
(iii) from about 10 mM to 100 mM of arginine or potassium tetraborate buffer; and optionally
(iv) a pH adjuster that is potassium hydroxide.

25. (canceled)

26. A method of treating congestion, edema, fluid overload, or hypertension in a subject in need thereof comprising administering to the subject the liquid pharmaceutical formulation of claim 1.

27. The method of claim 26, wherein the liquid pharmaceutical formulation is administered to the patient by subcutaneous injection.

28.-30. (canceled)

31. The method of claim 26, wherein the subject has chronic heart failure or chronic kidney disease.

32. (canceled)

33. A kit for the treatment of congestion, edema, fluid overload, or hypertension comprising the unit liquid pharmaceutical formulation of claim 23.

34. The kit of claim 33, comprising one, two, three or more units.

35. (canceled)

36. The kit of claim 33, further comprising an auto-injector or a patient-controlled injector.

37. (canceled)
